(19) United States
(12) Patent Application Publication
(10) Pub. No.: US 2025/0166793 A1
(43) Pub. Date: May 22, 2025

(54) PROBABILISTIC HR PREDICTION FOR COMPUTED TOMOGRAPHY

(71) Applicant: GE Precision Healthcare LLC, Waukesha, WI (US)

(72) Inventors: John Londt, Oconomowoc, WI (US); Arka Datta, Pewaukee, WI (US); Darin Okerlund, Muskego, WI (US)

(21) Appl. No.: 18/516,762

(22) Filed: Nov. 21, 2023

(51) Int. Cl. G16H 30/20; G06N 3/042; G16H 40/63

(52) U.S. Cl. CPC G16H 30/20; G06N 3/042; G16H 40/63

(57) ABSTRACT

Methods and systems are provided to predict an upper threshold and a lower threshold of a heart rate (HR) of a patient of a computed tomography (CT) imaging system using a machine learning (ML) model, based on HR time series data collected over a duration prior to a cardiac scan. The ML model takes as an additional input a desired probabilistic certainty (e.g., statistical confidence level) that the predicted upper and lower HR thresholds will be accurate, provided by an operator of the CT imaging system. Before the start of the imaging scan, the predicted upper and lower HR thresholds are used to set the start exposure and end exposure times, to ensure that the requested cardiac phases are acquired.

### TECHNICAL FIELD

Embodiments of the subject matter disclosed herein relate computerized tomography (CT) imaging systems, and in particular, to cardiac CT scans.

### BACKGROUND

In computed tomography (CT) imaging systems, an electron beam generated by a cathode is directed towards a target within an x-ray tube. A fan-shaped or cone-shaped beam of x-rays produced by electrons colliding with the target is directed towards a subject, such as a patient. After being attenuated by the object, the x-rays impinge upon an array of radiation detector elements. An electrical signal is generated at each detector element, and the electrical signals generated at the detector elements are used to reconstruct an image of the object, where each electrical signal corresponds to a voxel/pixel of the image.

Computed tomography is frequently employed to image a patient's heart. However, because the gantry requires time (e.g. one-half second) to make a full rotation, the continuous movement of the heart and the blood blurs the resultant images. Even a faster half-scan technique may still suffer from motion artifacts. Thus, it is desirable to minimize the amount of time required to generate each image slice to minimize motion related image degradation. For cardiac CT scans, because the quality of a reconstructed image of a heart of the subject may depend on a target portion of the heart moving as little as possible during a scan, cardiac scans may be performed at very short durations, when the heart is relatively still. To accomplish this, a target heart rate (HR) of a subject may first be predicted, for example, based on HR data collected during a pre-scan period, and the predicted target HR may be used to configure the CT system to start and end the cardiac scan at during a desired phase range of a cardiac cycle, such as in the middle of diastole of the cardiac cycle or end of systole of the cardiac cycle. However, because variance in a HR may be high, predicting the target HR with precision may be difficult. If the predicted target HR is inaccurate, the scan may be performed when the heart is moving, and the scan may fail.

### SUMMARY

In one example, a method for a computed tomography (CT) imaging system comprises predicting an upper threshold value and a lower threshold value of a heart rate (HR) of a patient of the CT imaging system, based on HR time series data collected from the patient over a duration and a confidence level specified by an operator of CT imaging system; configuring the CT imaging system to perform a cardiac scan during a cardiac phase range, based on the upper threshold value and the lower threshold value; performing the cardiac scan; reconstructing an image based on data acquired during the cardiac scan; and displaying the image on a display device of the CT imaging system and/or storing the image in a memory of the CT imaging system.

The above advantages and other advantages, and features of the present description will be readily apparent from the following Detailed Description when taken alone or in connection with the accompanying drawings. It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

Various aspects of this disclosure may be better understood upon reading the following detailed description and upon reference to the drawings in which:

FIG. 1 shows a pictorial view of a computed tomography (CT) imaging system, in accordance with one or more embodiments of the present disclosure;

FIG. 2 shows a block schematic diagram of an example CT imaging system, in accordance with one or more embodiments of the present disclosure;

FIG. 3 is a schematic diagram of an exemplary HR prediction system used by the CT imaging system, in accordance with one or more embodiments of the present disclosure;

FIG. 4A is a high-level information flow diagram showing a training system for training an HR time series prediction model, according to embodiments of the present disclosure;

FIG. 4B is a high-level information flow diagram showing a deployment of a trained HR time series prediction model during a scan performed using the CT imaging system, according to embodiments of the present disclosure;

FIG. 5 is a flowchart illustrating a method for training the HR time series prediction model, according to embodiments of the present disclosure;

FIG. 6 is a flowchart illustrating a method for using an HR time series prediction model to configure CT imaging system during a cardiac scan of a patient, according to embodiments of the present disclosure;

FIG. 7 is a graph of HR time series data showing a target scanning window, according to embodiments of the present disclosure;

FIG. 8 is an exploded portion of the graph of FIG. 7, according to embodiments of the present disclosure;

FIG. 9 shows an exemplary array of HR data values extracted from HR time series data, according to embodiments of the present disclosure;

FIG. 10 shows an example of HR time series windows extracted from the array of HR data values, according to embodiments of the present disclosure; and

FIG. 11 shows a comparison of various exemplary X-ray exposure times, according to embodiments of the present disclosure.

### DETAILED DESCRIPTION

This description and embodiments of the subject matter disclosed herein relate to using computed tomography (CT) imaging systems for cardiac imaging. In CT imaging systems in general, an X-ray source or X-ray tube emits an X-ray beam towards an object, such as a patient, and X-rays attenuated by the subject are detected by one or more detectors (e.g., a detector array) to generate projection data that is used to reconstruct one or more images. The X-ray detector or detector array typically includes a collimator for collimating X-ray beams received at the detector, a scintillator disposed adjacent to the collimator for converting X-rays to light energy, and photodiodes for receiving the light energy from the adjacent scintillator and producing electrical signals therefrom. An intensity of the attenuated X-ray beam radiation received at the detector array is typically dependent upon the attenuation of the X-ray beam by the patient. Each detector element of a detector array produces a separate electrical signal indicative of the attenuated beam received by each detector element. The electrical signals are transmitted to a data processing system for analysis. The data processing system processes the electrical signals to facilitate generation of an image. Generally, in CT systems the X-ray source and the detector array are rotated about a gantry within an imaging plane and around the patient, and images are generated from projection data at a plurality of views at different view angles. For example, for one rotation of the X-ray source, 1000 views may be generated by the CT system.

Heart activity is characterized by two distinct periods called systole and diastole. During systole, the heart muscle is contracting the volume of the left ventricle to pump the contents out through the aortic valve. At the end of the systole, the left ventricle has its smallest volume since it has been contracted to pump blood out. During the diastole, or the diastolic period, the left ventricle is filling through the mitral valve. The end of the diastole is the point at which the left ventricle has its largest volume since it is filled with blood ready to be pumped out. For the duration of the diastolic period, the heart is relatively motion-free, allowing images generated from data collected during this period to be clearer as a result of the limited movement.

In a typical CT cardiac imaging procedure, an examination starts with a patient laying on a table of a CT imaging system and being connected to an electrocardiograph (EKG). The EKG senses electrical activity of the heart and produces a cardiac wave form. The cardiac wave form is utilized to determine a heart rate (HR) of the patient prior to performing a cardiac scan using the CT imaging system. The CT imaging system is then typically operated to produce a “scout” scan of the patient, which is a preliminary scan of short duration to define the limits of table travel and other parameters to ensure that the heart will be imaged completely during the subsequent actual CT scan. During the scout scan (and/or other pre-contrast scans), a target HR of the patient is estimated using the EKG and stored in a memory of the CT imaging system. In addition, mean, standard deviation, minimum and maximum values for the HR, and a number of heart beats that occur during the pre-scan period may also be derived and stored.

In order to be able to distinguish blood vessels of the heart from surrounding tissue, a bolus of iodinated contrast material may be injected intravenously into the patient. The bolus is a solution which provides contrast between the blood flowing through the heart and the heart tissue, thus enabling the blood vessels to be distinguished in the resultant image. The bolus requires a certain amount of time to be carried by the circulatory system from the injection site to the heart in sufficient quantity to provide the desired contrast. Therefore, prior to the actual CT scan, a timing bolus scan may be conducted to determine that travel time. It should be understood that the bolus is quickly removed from the blood by other organs of the heart and thus must be re-injected each time an image is to be produced. Thus, during the timing bolus scan, the CT system is operated and the technician injects the bolus and then observes the CT images to identify the amount of time required for the bolus to reach the heart in sufficient quantity to produce a high contrast image. This time period is entered into the CT imaging system for use during the actual cardiac scan. The type of bolus and the rate of injection may also be entered into the CT system, as well as patient attributes, such as gender, weight, age, ethnic background, etc. that affect the HR and changes in the HR. HR data typically continues to be acquired prior to, during and after the timing bolus CT scan and recorded. In other cases, a different method may be used to determine a contrast injection timing.

Image quality is improved and motion artifacts are reduced by collecting x-ray attenuation data during a resting period of the heart. Although the activation of the x-ray beam can be dynamically synchronized to each cardiac cycle that occurs during the scan, other parameters such as gantry speed, table speed, and slice thickness must be defined prior to the scan and cannot be changed during the scan. The correct combination of these parameters is important to yield images with minimal motion artifacts, as their selection determines whether the x-ray source and detector will be properly located during diastolic period of each cardiac cycle. Hence, a quality of an image of the heart may rely on an accurate prediction of the patient's HR and the range of HR variation that will occur during the cardiac scan. The patient's HR may be predicted based on HR data collected from the patient and the patient attributes, such as gender, weight, age, ethnic background, etc. that affect the HR and changes in the HR. Additionally, the HR prediction may be informed by statistical techniques such as regression analysis applied to data collected from a plurality of patients. However, due to a complex interaction of factors and a high degree of variation across both across individuals and within individuals, an accuracy of the prediction may still be less than desired.

Because prospectively-gated cardiac CT studies rely on predicting future heartbeat durations to minimize x-ray exposure while still robustly capturing a desired phase(s) of the heart cycle, CT systems typically expand the range of phases acquired to account for heart prediction uncertainty. A radiologist either modifies the range of phases or the amount of padding (i.e. HR variation allowance) to adjust for this uncertainty. However, the padding assumes an equal probability of having a faster or slower heartbeat, whereas the probability of the heartbeat being faster is greater than the probability of the heartbeat being slower. As a result, an accuracy of the HR prediction may be lower than desired.

To increase the accuracy of the HR prediction, methods and systems are proposed herein to predict an HR of a patient based on HR time series data collected over a duration during the pre-scan period, using an HR time series prediction model, such as a machine learning (ML) model. Specifically, the HR time series prediction model may output an upper HR threshold value and a lower HR threshold value. Before the start of the imaging scan, the predicted upper and lower HR threshold values may be used to set scan parameters (e.g. table speed, gantry speed, slice thickness, etc.) and start exposure and end exposure times for the scan. Further, in addition to the collected HR time series data, the HR time series prediction model may take as an additional input a desired statistical confidence level that the predicted upper and lower HR thresholds will be correct, where the desired statistical confidence level is provided by an operator of the CT imaging system. The ML model may output the predicted upper and lower HR thresholds based on the desired confidence level. In some embodiments, the ML model may take additional inputs, such as whether a contrast agent was administered, statistical features extracted from the collected HR time series data (mean, standard deviation, amplitude of R-peaks, t-waves, p-waves, etc.), a classification of the data by a classification model, and/or other parameters or associated data.

In other words, rather than setting a padding around a predicted HR to account for uncertainty, a user of a CT imaging system can set the desired confidence level (e.g. 95%, high/med/low, etc.), and the CT imaging system calculates a corresponding dose impact corresponding to the upper and lower HR thresholds corresponding to the desired confidence level. The upper and lower HR thresholds that meet the prescribed confidence level would be determined by the HR time series prediction model. Unlike previous approaches, the upper and lower HR thresholds may not be symmetric with respect to a target HR of the subject. In this way, a quality of the HR prediction and the quality of the scan protocol selection may be advantageously increased.

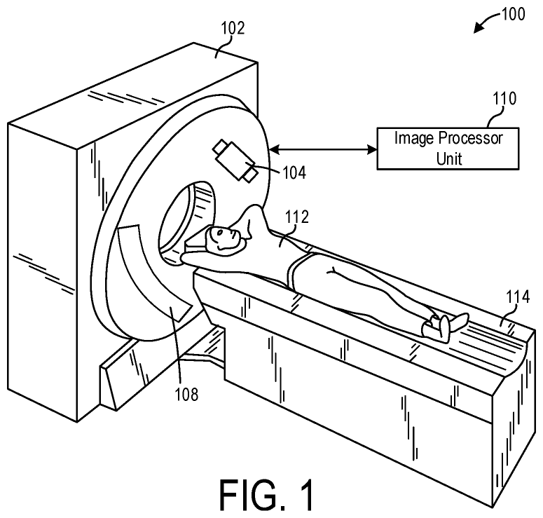

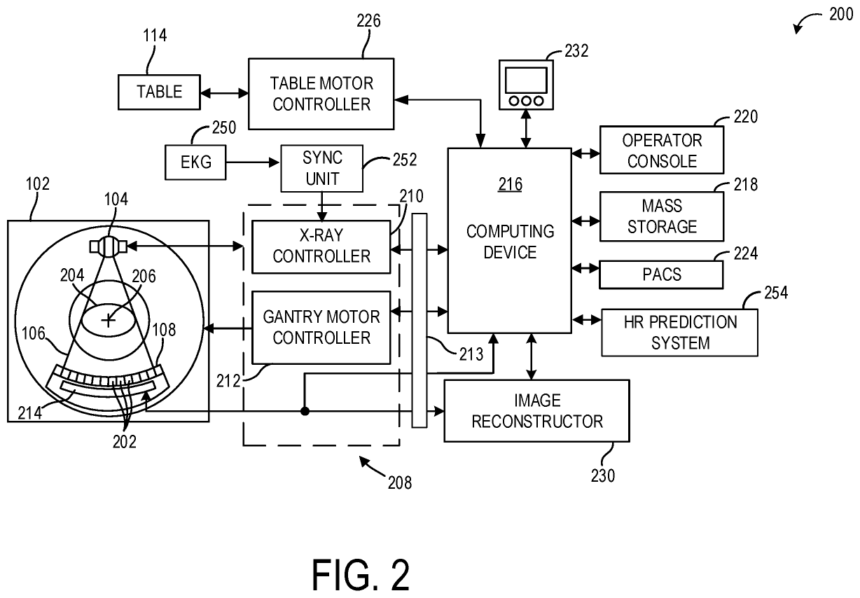

FIGS. 1-2 show example configurations with relative positioning of the various components. If shown directly contacting each other, or directly coupled, then such elements may be referred to as directly contacting or directly coupled, respectively, at least in one example. Similarly, elements shown contiguous or adjacent to one another may be contiguous or adjacent to each other, respectively, at least in one example. As an example, components laying in face-sharing contact with each other may be referred to as in face-sharing contact. As another example, elements positioned apart from each other with only a space there-between and no other components may be referred to as such, in at least one example. As yet another example, elements shown above/below/underneath one another, at opposite sides to one another, or to the left/right of one another may be referred to as such, relative to one another. Further, as shown in the figures, a topmost element or point of element may be referred to as a “top” of the component and a bottommost element or point of the element may be referred to as a “bottom” of the component, in at least one example. As used herein, top/bottom, upper/lower, above/below, may be relative to a vertical axis of the figures and used to describe positioning of elements of the figures relative to one another. As such, elements shown above other elements are positioned vertically above the other elements, in one example. As yet another example, shapes of the elements depicted within the figures may be referred to as having those shapes (e.g., such as being circular, straight, planar, curved, rounded, chamfered, angled, or the like). Further, elements shown intersecting one another may be referred to as intersecting elements or intersecting one another, in at least one example. Further still, an element shown within another element or shown outside of another element may be referred as such, in one example.

FIG. 1 illustrates an exemplary CT system 100 configured for CT imaging with photon-counting detectors. Particularly, the CT system 100 is configured to image a subject 112 such as a patient, an inanimate object, one or more manufactured parts, and/or foreign objects such as dental implants, stents, and/or contrast agents present within the body. The CT system 100 includes a gantry 102, which in turn, may further include at least one X-ray source 104 configured to project a beam of X-ray radiation 106 (see FIG. 2) for use in imaging the subject 112 laying on a table 114. Specifically, the X-ray source 104 is configured to project the X-ray radiation beams 106 towards a detector array 108 positioned on the opposite side of the gantry 102. Although FIG. 1 depicts a single X-ray source 104, in certain embodiments, multiple X-ray sources and detectors may be employed to project a plurality of X-ray radiation beams for acquiring projection data at the same or different energy levels corresponding to the patient. In some embodiments, the X-ray source 104 may enable dual-energy gemstone spectral imaging (GSI) by rapid peak kilovoltage (kVp) switching. In the embodiments described herein, the X-ray detector employed is a photon-counting detector which is capable of differentiating X-ray photons of different energies.

In certain embodiments, the CT system 100 further includes an image processor unit 110 configured to reconstruct images of a target volume of the subject 112 using an iterative or analytic image reconstruction method. For example, the image processor unit 110 may use an analytic image reconstruction approach such as filtered back projection (FBP) to reconstruct images of a target volume of the patient. As another example, the image processor unit 110 may use an iterative image reconstruction approach such as advanced statistical iterative reconstruction (ASIR), conjugate gradient (CG), maximum likelihood expectation maximization (MLEM), model-based iterative reconstruction (MBIR), and so on to reconstruct images of a target volume of the subject 112. In some examples the image processor unit 110 may use an analytic image reconstruction approach, such as FBP, in addition to an iterative image reconstruction approach.

In some CT imaging system configurations, an X-ray source projects a cone-shaped X-ray radiation beam which is defined with respect to an X-Y-Z Cartesian coordinate system and generally referred to as an “imaging volume.” The X-ray radiation beam passes through an object being imaged, such as the patient or subject. The X-ray radiation beam, after being attenuated by the object, impinges upon an array of detector elements. The intensity of the attenuated X-ray radiation beam received at the detector array is dependent upon the attenuation of an X-ray radiation beam by the object. Each detector element of the array produces a separate electrical signal that is a measurement of the X-ray beam attenuation at the detector location. The attenuation measurements from all the detector elements are acquired separately to produce a transmission profile.

In some CT systems, the X-ray source and the detector array are rotated with a gantry within the imaging volume and around the object to be imaged such that an angle at which the X-ray beam intersects the object constantly changes. A group of X-ray radiation attenuation measurements, e.g., projection data, from the detector array at one gantry angle is referred to as a “view.” A “scan” of the object includes a set of views made at different gantry angles, or view angles, during one revolution of the X-ray source and detector.

FIG. 2 illustrates an exemplary imaging system 200 similar to the CT system 100 of FIG. 1. In accordance with aspects of the present disclosure, the imaging system 200 is configured for imaging a subject 204 (e.g., the subject 112 of FIG. 1). In one embodiment, the imaging system 200 includes the detector array 108 (see FIG. 1). The detector array 108 further includes a plurality of detector elements 202 that together sense the X-ray radiation beam 106 (see FIG. 2) that passes through the subject 204 (such as a patient) to acquire corresponding projection data. In some embodiments, the detector array 108 may be fabricated in a multi-slice configuration including the plurality of rows of cells or detector elements 202, where one or more additional rows of the detector elements 202 are arranged in a parallel configuration for acquiring the projection data.

In certain embodiments, the imaging system 200 is configured to traverse different angular positions around the subject 204 for acquiring desired projection data. Accordingly, the gantry 102 and the components mounted thereon may be configured to rotate about a center of rotation 206 for acquiring the projection data, for example, at different energy levels. Alternatively, in embodiments where a projection angle relative to the subject 204 varies as a function of time, the mounted components may be configured to move along a general curve rather than along a segment of a circle.

As the X-ray source 104 and the detector array 108 rotate, the detector array 108 collects data of the attenuated X-ray beams. The data collected by the detector array 108 undergoes pre-processing and calibration to condition the data to represent the line integrals of the attenuation coefficients of the scanned subject 204. The processed data are commonly called projections. In some examples, the individual detectors or detector elements 202 of the detector array 108 may include photon-counting detectors which register the interactions of individual photons into one or more energy bins.

The acquired sets of projection data may be used for basis material decomposition (BMD). During BMD, the measured projections are converted to a set of material-density projections. The material-density projections may be reconstructed to form a set of material-density maps or images of each respective basis material, such as bone, soft tissue, and/or contrast agent maps. The density maps or images may be, in turn, associated to form a 3D volumetric image of the basis material, for example, bone, soft tissue, and/or contrast agent, in the imaged volume.

Once reconstructed, the basis material image produced by the imaging system 200 reveals internal features of the subject 204, expressed in the densities of two basis materials. The density image may be displayed to show these features. In traditional approaches to diagnosis of medical conditions, such as disease states, and more generally of medical events, a radiologist or physician would consider a hard copy or display of the density image to discern characteristic features of interest. Such features might include lesions, sizes and shapes of particular anatomies or organs, and other features that would be discernable in the image based upon the skill and knowledge of the individual practitioner.

In one embodiment, the imaging system 200 includes a control mechanism 208 to control movement of the components such as rotation of the gantry 102 and the operation of the X-ray source 104. In certain embodiments, the control mechanism 208 further includes an X-ray controller 210 configured to provide power and timing signals to the X-ray source 104. Additionally, the control mechanism 208 includes a gantry motor controller 212 configured to control a rotational speed and/or position of the gantry 102 based on imaging requirements.

In certain embodiments, the control mechanism 208 further includes a data acquisition system (DAS) 214 configured to sample analog data received from the detector elements 202 and convert the analog data to digital signals for subsequent processing. The DAS 214 may be further configured to selectively aggregate data from a subset of the detector elements 202 into so-called macro-detectors. The data sampled and digitized by the DAS 214 may be transmitted to a computer or computing device 216 via a slip ring 213. In one example, the computing device 216 stores the data in a storage device or mass storage 218. The storage device 218, for example, may be any type of non-transitory memory and may include a hard disk drive, a floppy disk drive, a compact disk-read/write (CD-R/W) drive, a Digital Versatile Disc (DVD) drive, a flash drive, and/or a solid-state storage drive.

Additionally, the computing device 216 provides commands and parameters to one or more of the DAS 214, the X-ray controller 210, and the gantry motor controller 212 for controlling system operations such as data acquisition and/or processing. In certain embodiments, the computing device 216 controls system operations based on operator input. The computing device 216 receives the operator input, for example, including commands and/or scanning parameters via an operator console 220 operatively coupled to the computing device 216. The operator console 220 may include a keyboard (not shown) or a touchscreen to allow the operator to specify the commands and/or scanning parameters.

Although FIG. 2 illustrates one operator console 220, more than one operator console may be coupled to the imaging system 200, for example, for inputting or outputting system parameters, requesting examinations, plotting data, and/or viewing images. Further, in certain embodiments, the imaging system 200 may be coupled to multiple displays, printers, workstations, and/or similar devices located either locally or remotely, for example, within an institution or hospital, or in an entirely different location via one or more configurable wired and/or wireless networks such as the Internet and/or virtual private networks, wireless telephone networks, wireless local area networks, wired local area networks, wireless wide area networks, wired wide area networks, etc.

In one embodiment, for example, the imaging system 200 either includes, or is coupled to, a picture archiving and communications system (PACS) 224. In an exemplary implementation, the PACS 224 is further coupled to a remote system such as a radiology department information system, hospital information system, and/or to an internal or external network (not shown) to allow operators at different locations to supply commands and parameters and/or gain access to the image data.

The computing device 216 uses the operator-supplied and/or system-defined commands and parameters to operate a table motor controller 226, which in turn, may control a table 114 which may be a motorized table. Specifically, the table motor controller 226 may move the table 114 for appropriately positioning the subject 204 in the gantry 102 for acquiring projection data corresponding to the target volume of the subject 204.

As previously noted, the DAS 214 samples and digitizes the projection data acquired by the detector elements 202. Subsequently, an image reconstructor 230 uses the sampled and digitized X-ray data to perform high-speed reconstruction. Although FIG. 2 illustrates the image reconstructor 230 as a separate entity, in certain embodiments, the image reconstructor 230 may form part of the computing device 216. Alternatively, the image reconstructor 230 may be absent from the imaging system 200 and instead the computing device 216 may perform one or more functions of the image reconstructor 230. Moreover, the image reconstructor 230 may be located locally or remotely, and may be operatively connected to the imaging system 200 using a wired or wireless network. Particularly, one exemplary embodiment may use computing resources in a “cloud” network cluster for the image reconstructor 230.

In one embodiment, the image reconstructor 230 stores the images reconstructed in the storage device 218. Alternatively, the image reconstructor 230 may transmit the reconstructed images to the computing device 216 for generating useful patient information for diagnosis and evaluation. In certain embodiments, the computing device 216 may transmit the reconstructed images and/or the patient information to a display or display device 232 communicatively coupled to the computing device 216 and/or the image reconstructor 230. In some embodiments, the reconstructed images may be transmitted from the computing device 216 or the image reconstructor 230 to the storage device 218 for short-term or long-term storage.

Information may be transmitted between the components residing in the gantry 102 and external devices (such as the computing device 216 and/or image reconstructor 230) via the slip ring 213, which facilitates electronic communication across the rotating gantry.

For cardiac scans, an electrocardiograph (EKG) may be utilized to sense cardiac activity of the patient. EKG 250 may be of a conventional design that utilizes electrodes attached to the patient's chest to detect electrical activity of a heart. The electrical activity is represented by a standard cardiac waveform signal produced at an output of the EKG 250. A portion of the signal known as the QRS complex contains the R-wave, which is the most prominent, highest amplitude, feature of the entire signal, and the cardiac cycle is typically defined as beginning with an R-wave and continuing until the occurrence of the next R-wave.

The output signal from the EKG 250 is applied to a synchronization (SYNC) unit or circuit 252, which interfaces to x-ray controller 210. The signal received from synchronization unit 252 enables x-ray controller 210 to synchronize production of short bursts of x-rays during a selected portion of a cardiac cycle of a patient. Specifically, the cardiac waveform signal is used to determine a timing and duration of data collection. In one embodiment, synchronization unit 252 activates x-ray emission after delaying a selected period of time from commencement of a cardiac cycle, so that an x-ray beam is emitted during the selected period of the cardiac cycle. X-ray attenuation data collected from a series of these short bursts of x-rays may then be utilized to generate an image of the heart by conventional backprojection methods.

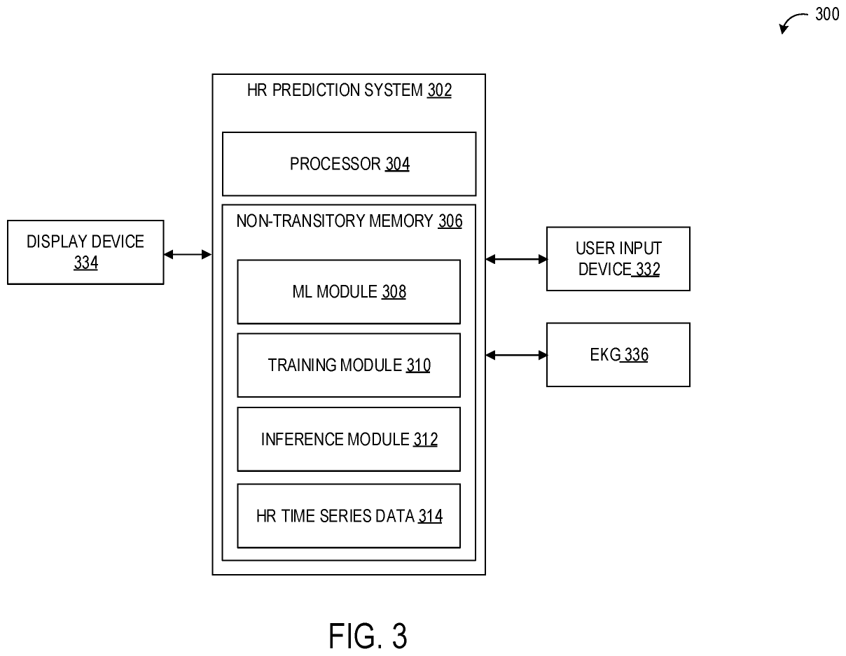

Imaging system 200 further includes an HR prediction system 254. Because image quality is improved and motion artifacts are reduced by collecting x-ray attenuation data during a resting period of the heart, a quality of a cardiac scan may rely on an accurate prediction of the patient's HR and the range of HR variation that will occur during the cardiac scan. HR prediction system 254 may be used by x-ray controller 210 in conjunction with SYNC unit 252 to determine the timing and duration of the data collection. HR prediction system 254 is described in greater detail below in reference to FIG. 3.

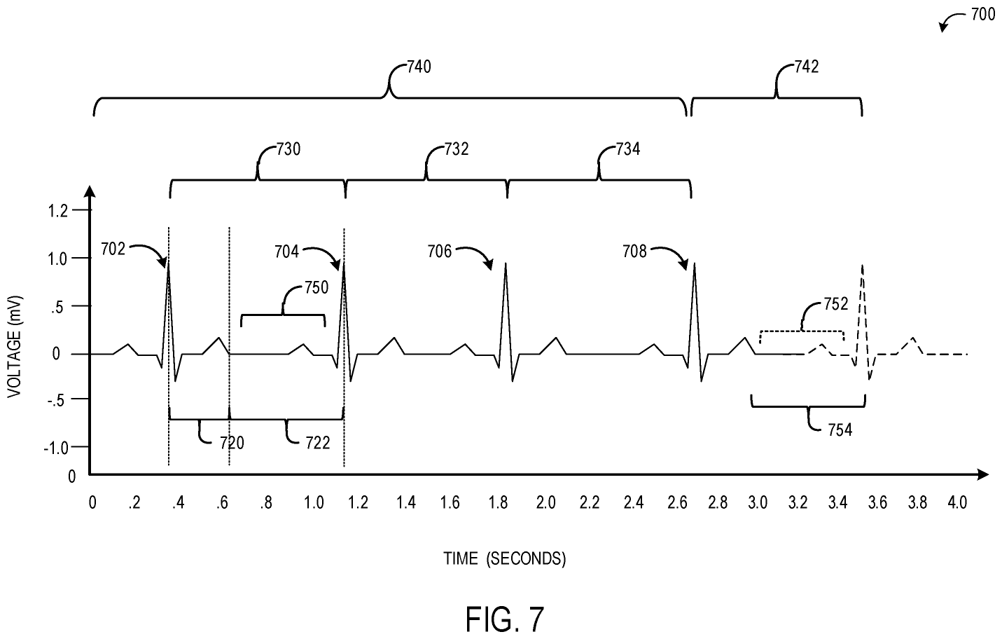

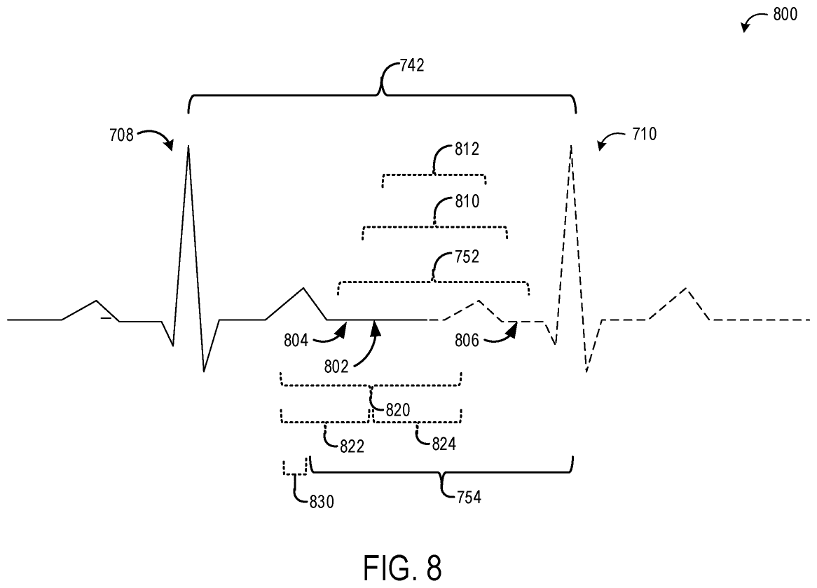

FIGS. 7 and 8 provide an overview of how HR prediction system 254 may be used in conjunction with imaging system 200. FIG. 7 shows a time series graph 700 of a HR of a patient as a cardiac scan is being performed using an imaging system, such as imaging system 200 of FIG. 2. The HR may be detected by a monitoring device, such as EKG 250 imaging system 200.

Time series graph 700 shows various cardiac cycles, where each cardiac cycle of the various cardiac cycles begins and ends with a heartbeat. A first cardiac cycle 730 begins with a first heartbeat 702 and ends with a second heartbeat 704; a second cardiac cycle 732 begins with second heartbeat 704 and ends with a third heartbeat 706; and a third cardiac cycle 734 begins with third heartbeat 706 and ends with a fourth heartbeat 708. Each heartbeat is characterized by an increase in blood pressure as the heart contracts to push blood out to a body of the patient during a systolic portion 720 of the cardiac cycle, and a subsequent decrease in blood pressure as the heart relaxes to fill with blood received from veins of the body during a diastolic portion 722 of the cardiac cycle.

For a patient with a normal heart, each cardiac cycle of the patient may have a slightly different duration, due to normal variability in the HR of the patient. For example, first cardiac cycle 730 may have a first duration; second cardiac cycle 732 may have a second duration, where the second duration is shorter or longer than the first duration; and third cardiac cycle 734 may have a third duration, where the third duration may be shorter or longer than either or both of the first duration and second duration. In the depicted example, second cardiac cycle 732 has a slightly shorter duration than first cardiac cycle 730, and third cardiac cycle 734 has a slightly longer duration than first cardiac cycle 730.

A quality of the cardiac scan may depend on imaging the heart of the time when an amount of motion of the heart is minimized, meaning, when the heart is at rest and the amount of motion is low. If the cardiac scan is performed at a time when the heart is moving, an image resulting from the cardiac scan may be blurred and/or may include artifacts that render the image unusable for diagnostic and/or therapeutic reasons. In such cases, one or more subsequent scans may be performed to generate a higher quality image. Because each subsequent scan may increase the dose of radiation to which the patient is exposed, it is desirable to perform as few scans as possible to achieve an acceptable image. To perform the scan when the amount of motion of the heart is minimized, the imaging system may be configured to image the heart during a specified cardiac phase range 750 of the cardiac cycle (e.g., mid-diastole or end-systole), when the heart is relaxed and slowly filling with blood. In various embodiments, the specified cardiac phase range 750 may be prescribed by an operator of the CT imaging system at a time of a cardiac scan. The cardiac phase range may be specified in a variety of ways, such as, for example, R-peak to R-peak, milliseconds prior to or after an R-peak, or other ECG waveform features could be used, such as P-wave or T-wave.

In order to configure the imaging system to image the heart during cardiac phase range 750, a moving window 740 of time series HR data may be analyzed by a HR prediction system (e.g., HR prediction system 254). Moving window 740 may comprise a plurality of cardiac cycles. In FIG. 7, moving window 740 comprises first cardiac cycle 730, second cardiac cycle 732, and third cardiac cycle 734. Based on the time series HR data included in moving window 740, an HR of the patient over a duration (e.g., 10 seconds) may be predicted by the HR prediction system. The HR may be defined by an upper threshold HR value, and a lower threshold HR value. For example, the upper threshold HR value may be 75, and the lower threshold HR value may be 64.

Based on the predicted upper and lower threshold HR values and the prescribed cardiac phase range 750, a target scanning duration 752 of a predicted diastolic portion 754 of a predicted future cardiac cycle 742 may be calculated, where target scanning duration 752 establishes a window of time during which the cardiac scan may be performed with a degree of confidence that the amount of motion of the heart will be below the threshold amount of motion. In some embodiments, a start of the window of time during which the cardiac scan may be performed may be configured based on the upper threshold HR value, and an end of the window of time may be configured based on the lower threshold HR value.

FIG. 8 shows an expanded portion 800 of time series graph 700, where expanded portion 800 includes predicted cardiac cycle 742. As described above in reference to FIG. 7, predicted target scanning duration 752 occurs within diastolic portion 754 of predicted cardiac cycle 742, when the heart of the patient is resting. As described above, predicted target scanning duration 752 may establish a window of time during which the cardiac scan may be performed with a first degree of confidence that the scan will be performed during the prescribed cardiac phase range 750. It should be noted that some types of cardiac scans can tolerate more motion of the heart than other types of cardiac scans. For example, a first, greater amount of motion of the heart may be tolerated during a scan of an aorta, and a second, smaller amount of motion of the heart may be tolerated during a scan of a valve of the heart. As a result, the window of time during which the cardiac scan may be performed may be longer when scanning the aorta than when scanning the valve.

Predicted target scanning duration 752 represents a range of times during which the scan may be performed in accordance with the user prescribed cardiac phase range 750. A probability that the motion of the heart will be minimal may be highest at a time 802 within the range. The probability may decrease for times that are closer to an edge of predicted target scanning duration 752 (e.g., closer to one end of the prescribed cardiac phase range). For example, the probability may be lower at times 804 and 806 than during a time window 810.

In other words, a HR time series prediction model used by the HR prediction system to predict upper and lower threshold values of an HR of the patient may have a highest level of confidence that target scanning duration 752 is aligned with or within the prescribed cardiac phase range 750 at time 802, and the level of confidence of the HR time series prediction model may be lower at times 804 and 806. Thus, predicted target scanning duration 752 may be selected based on a desired level of confidence that the motion of the heart will be minimized. When scanning the aorta (or a different structure of the heart more tolerant to motion during a scan), a first lower level of confidence may be specified (e.g., by an operator of the imaging system) to generate a first predicted target scanning duration of a first length. When scanning the valve, a second, higher level of confidence may be specified to generate a second predicted target scanning duration of a second length, where the second length is smaller than the first length.

As an example, when configuring the imaging system to perform the cardiac scan of the aorta, a radiologist may specify that the first predicted target scanning duration should represent a first range of times during which the model has a 65% confidence level that the motion of the heart will be minimized. The first predicted target scanning duration may be equal to predicted target scanning duration 752 of FIG. 8. To perform a second cardiac scan of the valve, the radiologist may specify that the second predicted target scanning duration should represent a second range of times during which the model has a 95% confidence level that the motion of the heart will be minimized. The second predicted target scanning duration may be equal to a predicted target scanning duration 812, where second predicted target scanning duration 812 is less than predicted target scanning duration 752. Thus, the radiologist can specify how important it is for a given cardiac scan for the motion of the heart to be minimized by specifying the confidence level, and the HR time series prediction model may calculate a starting time and an ending time of a range of times during which a probability of the motion of the heart being minimized, and output a corresponding predicted target scanning duration to be used to configure the imaging system to perform the cardiac scan.

However, it should be appreciated that time 802 may not be at a middle of the range of times represented by predicted target scanning duration 752 during which the scan may be performed with minimal motion of the heart. This is because a first probability of the HR increasing may be higher than a second probability of the HR decreasing. If the HR increases, predicted target scanning duration 752 may decrease accordingly. Because of this asymmetry, time 802 may be closer to a beginning of the range of times. Because the HR time series prediction model predicts the predicted target scanning duration based on the specified confidence level, and not based on predicting a time, a quality of a first image generated using the HR time series prediction model may have a higher probability of being higher than a second image generated by conventional methods without using the HR time series prediction model. The conventional methods may include predicting a specific time at which the motion of the heart is lowest, and generating a range of times around the specific time by adding padding time symmetrically around the specific time.

For example, an alternative scanning duration 820 may be generated by an alternative HR prediction model, where alternative scanning duration 820 is based on a prediction that the motion of the heart will be lowest at time 802. A first padding time 822 and a second padding time 824 may be symmetrically applied around time 802 by the alternative HR prediction model to generate alternative scanning duration 820. However, in contrast to predicted target scanning durations 752, 810, and 812, alternative scanning duration 820 may include a portion 830 of a systolic portion of cardiac cycle 742, during which the heart may be moving. Thus, using alternative scanning duration 820 to configure the imaging system to perform the cardiac scan may result in a higher probability of images below the threshold quality, and a higher probability that additional scans may be performed.

Referring now to FIG. 3, a block diagram 300 shows an HR prediction system 302, in accordance with an embodiment. In some embodiments, HR prediction system 302 is incorporated into imaging system 200, as described above. In some embodiments, at least a portion of HR prediction system 302 is disposed at a device (e.g., edge device, server, etc.) communicably coupled to imaging system 200 via wired and/or wireless connections. In some embodiments, HR prediction system 302 may be operably/communicatively coupled to a user input device 332 and a display device 334. For example, user input device 332 may be included within operator console 220 of the imaging system 200, while display device 334 may be included within display device 232 of imaging system 200, at least in some examples.

HR prediction system 302 includes a processor 304 configured to execute machine readable instructions stored in non-transitory memory 306. Processor 304 may be single core or multi-core, and the programs executed thereon may be configured for parallel or distributed processing. In some embodiments, processor 304 may optionally include individual components that are distributed throughout two or more devices, which may be remotely located and/or configured for coordinated processing. In some embodiments, one or more aspects of processor 304 may be virtualized and executed by remotely-accessible networked computing devices configured in a cloud computing configuration.

Non-transitory memory 306 may store a machine learning (ML) module 308, a training module 310, an inference module 312, and HR time series data 314. ML module 308 may include one or more ML models, and instructions for implementing the one or more ML models to predict upper and lower threshold values of an HR of a patient of the imaging system, as described in greater detail below. ML module 308 may include models of various types, including trained and/or untrained neural networks such as CNNs, statistical models, or other models, and may further include various data, or metadata pertaining to the one or more models stored therein. In particular, ML module 308 may include an HR time series prediction model for predicting an HR of the patient based on HR data continuously collected during a preceding time series window.

In some embodiments, prediction HR system 302 may be operably/communicatively coupled to an EKG 336, which may provide HR data used for training the HR time series prediction model and/or for predicting an HR of a patient of the imaging system using a trained HR time series prediction model. EKG 336 may be a non-limiting example of EKG 250 of FIG. 2.

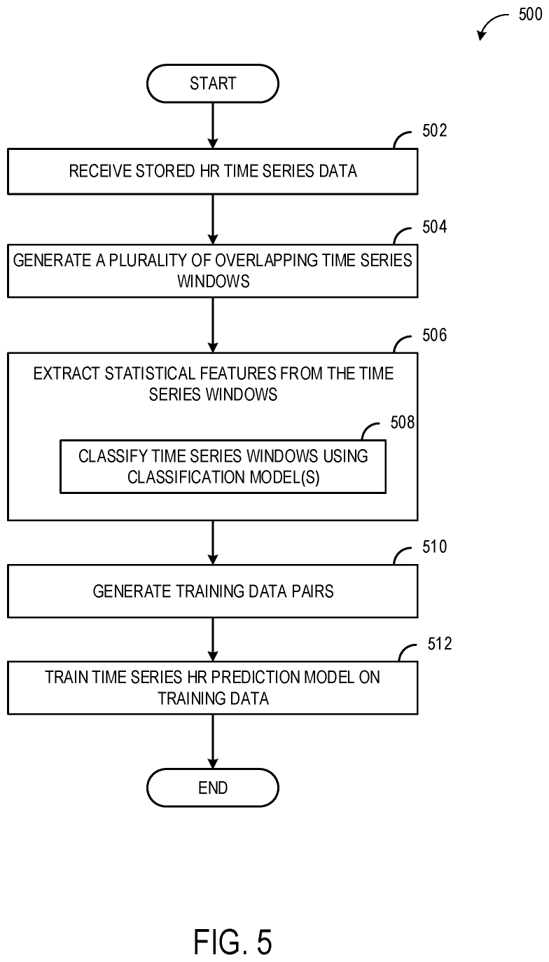

Non-transitory memory 306 may further store a training module 310, which may comprise instructions for training one or more of the models stored in ML module 308. In particular, training module 310 may include instructions that, when executed by processor 304, cause HR prediction system 302 to conduct one or more of the steps of method 500 for training a HR time series prediction model, described below in reference to FIG. 5. In some embodiments, training module 310 may include instructions for implementing one or more gradient descent algorithms, applying one or more loss functions, and/or training routines, for use in adjusting parameters of one or more ML models of ML module 308. Training module 310 may include training datasets for the one or more models of ML module 308.

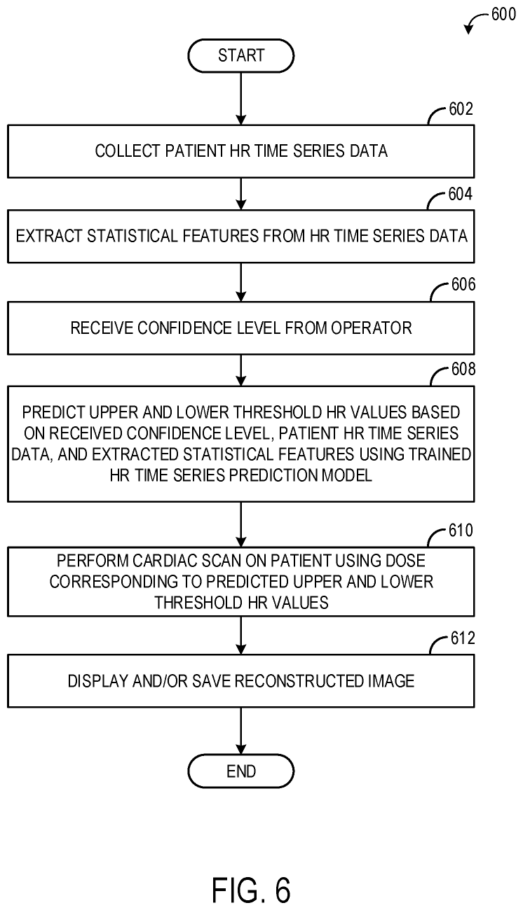

Non-transitory memory 306 also stores an inference module 312. Inference module 312 may include instructions for deploying a trained HR time series prediction model to predict upper and lower HR thresholds of a patient, as described below with respect to FIG. 6. In particular, inference module 312 may include instructions that, when executed by processor 304, cause HR prediction system 302 to conduct one or more of the steps of method 600.

Non-transitory memory 306 further stores HR time series data 314. HR time series data 314 may include, for example, HR data collected from a plurality of patients and stored in a database. For example, HR time series data 314 may include cardiac time series data, including HR data collected from one or more patients during a moving time series window. HR time series data 314 may include cardiac data used in one or more training sets for training the HR time series prediction model of ML module 308, or for predicting an HR of a patient using a trained HR time series prediction model.

In some embodiments, non-transitory memory 306 may include components disposed at two or more devices, which may be remotely located and/or configured for coordinated processing. In some embodiments, one or more aspects of non-transitory memory 306 may include remotely-accessible networked storage devices configured in a cloud computing configuration.

User input device 332 may comprise one or more of a touchscreen, a keyboard, a mouse, a trackpad, or other device configured to enable a user to interact with and manipulate data within HR prediction system 302. In one example, user input device 332 may enable a user to collect HR data from a patient of the imaging system, and/or select HR data from one or more patients of the imaging system for training purposes. Additionally, user input device 332 may enable a user to input a desired confidence level for an accuracy of an output of HR prediction system 302.

Display device 334 may include one or more display devices utilizing virtually any type of technology. In some embodiments, display device 334 may comprise a computer monitor. Display device 334 may be combined with processor 304, non-transitory memory 306, and/or user input device 332 in a shared enclosure, or may be peripheral display devices and may comprise a monitor, touchscreen, projector, or other display device known in the art.

It should be understood that HR prediction system 302 shown in FIG. 3 is for illustration, not for limitation. Another appropriate HR prediction system may include more, fewer, or different components. Additionally, while the systems and methods described herein refer to predicting an HR of a patient, the systems and methods may also be applied to predicting a heartbeat duration of the patient. For the purposes of this disclosure, the terms “heart rate (HR)” and “heartbeat duration” may be used interchangeably.

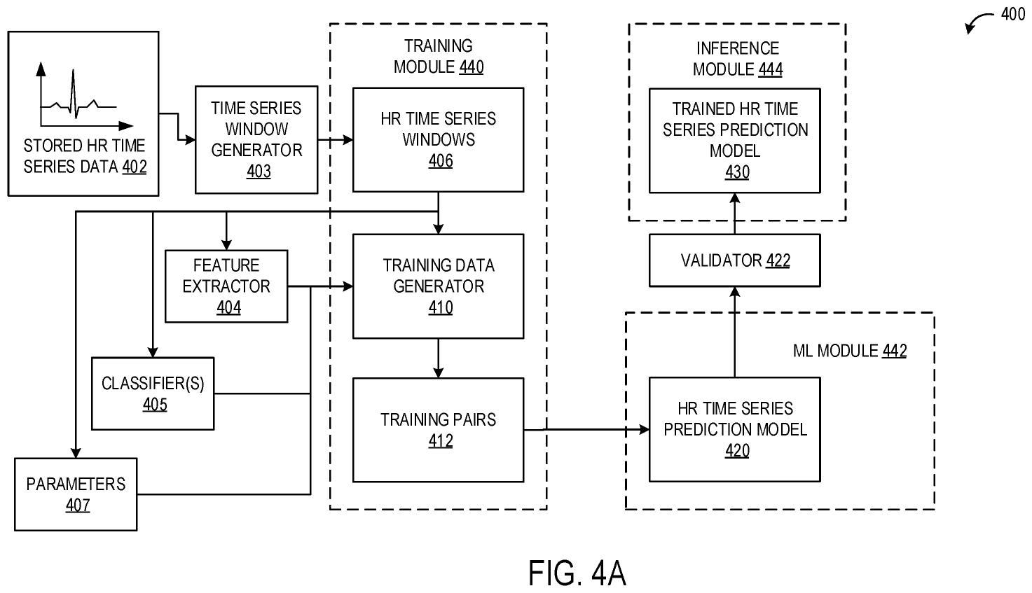

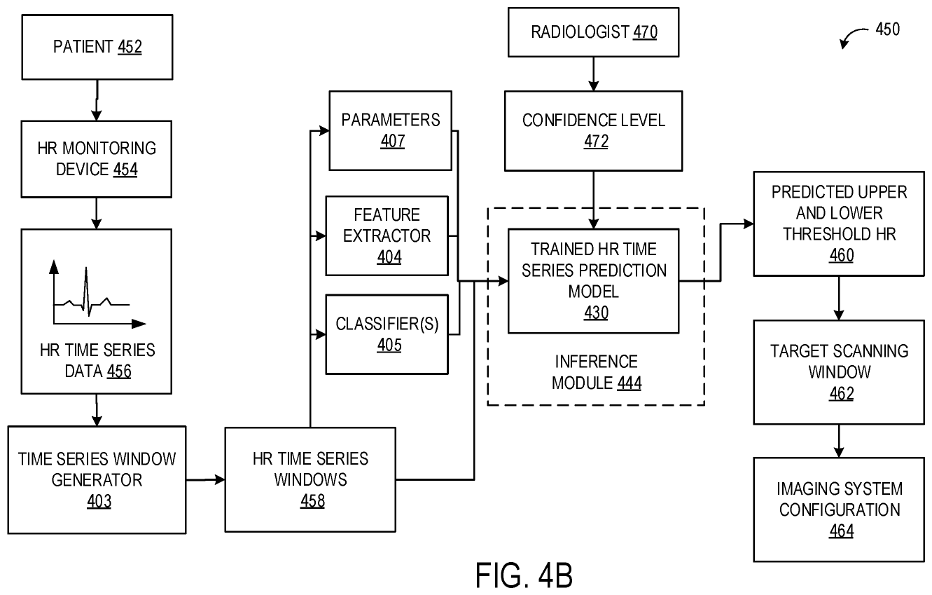

Referring to FIG. 4A, an exemplary HR time series prediction model training system 400 is shown, which may be used to train a HR time series prediction model 420. HR time series prediction model 420 may be trained to predict expected (target) upper and lower threshold values of an HR of a patient of an imaging system during a cardiac scan, such that a target anatomy of the heart of the patient can be scanned when the heart is at rest and motion of the heart is minimized.

HR time series prediction model 420 may be used by a HR prediction system, such as HR prediction system 302 of FIG. 3, in conjunction with an imaging system, such as imaging system 200 of FIG. 2. HR time series prediction model 420 may be stored within a ML model module 442 of the HR prediction system. ML model module 442 may be a non-limiting example of ML module 308 of HR prediction system 302 of FIG. 3.

HR time series prediction model 420 may be trained on stored HR time series data 402 collected from a plurality of human subjects. The plurality of human subjects may include subjects drawn from a wide population to generate a diverse set of HR data. For example, the plurality of human subjects may include subjects of different ages, different body types and sizes (including adults and children), and so on. The plurality of human subjects may also include healthy subjects and subjects suffering from one or more health conditions. In various embodiments, stored HR time series data 402 may be time series data represented as a continuous stream of changes in blood pressure, or voltage, over time. In various embodiments, stored HR time series data 402 may be collected from a variety of patients of a healthcare system, for example, captured via patient monitors using for monitoring the patients.

HR time series prediction model training system 400 may include a time series window generator 403, which may extract portions of stored HR time series data 402 corresponding to discrete time series windows 406. For example, time series window 406 may comprise 5 seconds of stored HR time series data 402, or 10 seconds of stored HR time series data 402, or a different amount of time. During training of HR time series prediction model 430, time series window generator 403 may continuously extract time series windows 406 from one or more subjects included in stored HR time series data 402. For example, stored HR time series data 402 may include a first set of HR time series data from a first subject; a second set of HR time series data from a second subject, a third set of HR time series data from a first subject; and so on. Durations of the first set of HR time series data, the second set of HR time series data, and the third set of HR time series data may be equal, or may not be equal.

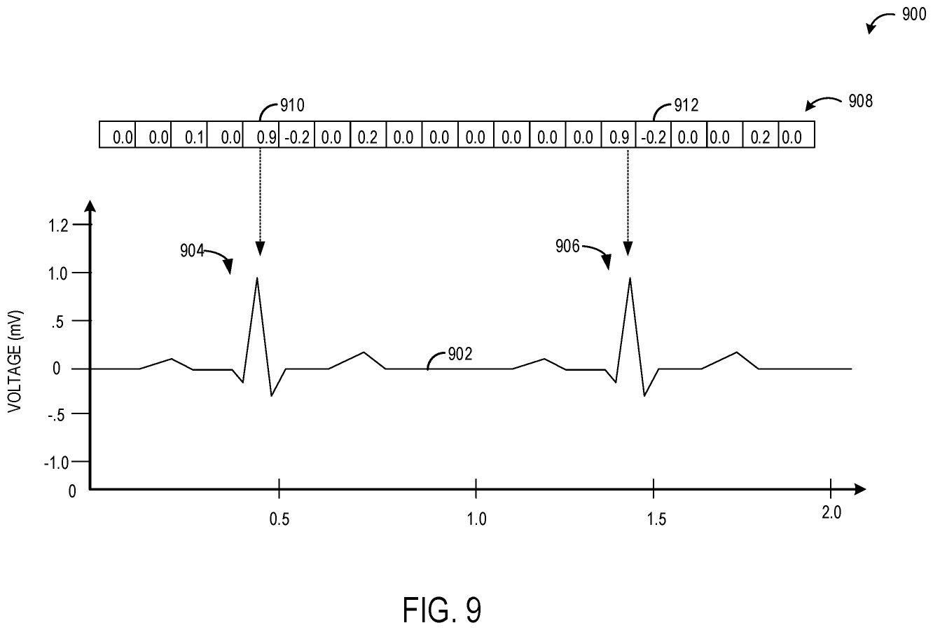

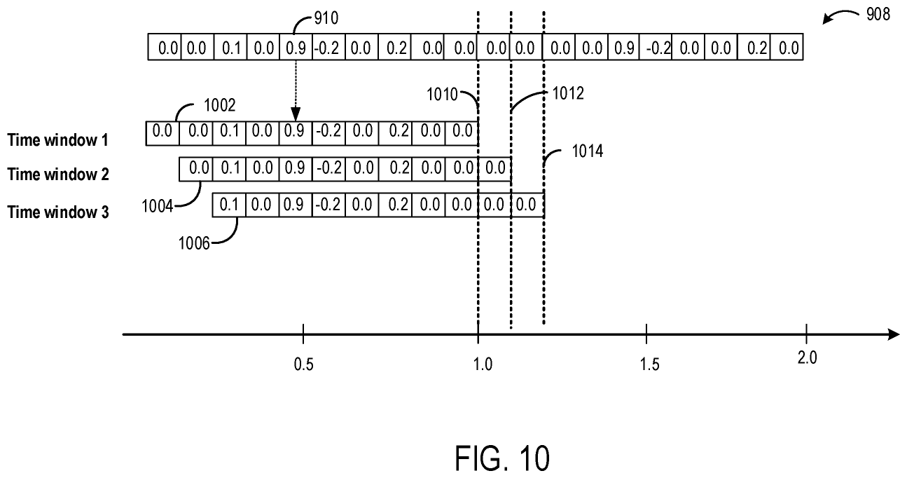

During training of HR time series prediction model 420, a first plurality of time series windows 406 may be extracted from the first set of HR time series data; a second plurality of time series windows 406 may be extracted from the second set of HR time series data; a third plurality of time series windows 406 may be extracted from the third set of HR time series data; and so on. Additionally, HR time series windows 406 may be overlapping time series windows, as described below in reference to FIGS. 9 and 10. The first plurality of time series windows 406, the second plurality of time series windows 406, and the third plurality of time series windows 406 may be used to train HR time series prediction model 420. In this way, HR time series prediction model 420 may be trained on time series HR data collected from a variety of subjects.

During extraction of the HR time series windows 406 from the stored HR time series data 402, portions of stored HR time series data 402 may be stored in a data array in a buffer in a memory of the HR prediction system (e.g., non-transitory memory 306 of HR prediction system 302), and each HR time series windows 406 may be selected from the data array based on an initial time reference, as described below in reference to FIGS. 5, 9, and 10.

HR time series prediction model training system 400 may include a feature extractor 404 and/or one or more classifiers 405, which may generate additional input data to be used for training HR time series prediction model 420. The feature extractor 404 may be a third-party EKG trigger device, or patient monitoring device of the CT imaging system. The feature extractor 404 may extract statistical features from the one or more time series windows 406, and the extracted statistical features may be inputs into HR time series prediction model 420 during training. The extracted statistical features may include, for example, an amplitude of an R-wave (R-peak) of a time series window 406, an amplitude and/or time of a p-wave of the time series window 406, an amplitude and/or time of a t-wave of the time series window 406, a slope of a QRS complex of a time series window 406, a quantification of electrocardiograph (EKG) noise in the time series window 406 using a Fourier analysis or other general signal processing techniques, or a different statistical feature (means, medians, standard deviations, etc.). The one or more classifiers 405 may include classification models of the CT imaging system that may classify a time series window 406 into one or more classifications. For example, the classification model may detect an abnormal heartbeat (e.g., atrial fibrillation (A-fib), bigeminy/trigeminy, etc.), and may classify the time series window based on detected abnormal heartbeat. The classification may be an additional input into HR time series prediction model 420 during training.

Additionally, parameters of the CT imaging system associated with the HR time series windows 406 may be additional inputs into the HR time series prediction model during training. The parameters may include, for example, whether a contrast agent was administered to a relevant patient of an HR time series window 406; contrast injection parameters (e.g., volume, rate, injection delay); a scan series delay; a breath hold duration and/or breath hold parameters (e.g., free-breathing, end inspiration, end expiration, etc.); or a different parameter.

HR time series prediction model training system 400 may include a training data generator 410, which may generate a plurality of training pairs 412. Training data generator may be stored in a training module 440 of the HR prediction system, such as training module 310 of HR prediction system 302 of FIG. 3. Each training pair 412 may include an HR time series window 406 of a subject as input data, and measured upper and lower bounds of a HR of the subject as ground truth data. For example, the HR time series window 406 may include a first array of data values of stored HR time series data 402 corresponding to a subject, where each data value of the first array of data values is a blood pressure reading at a predefined time interval. The ground truth data may comprise upper and lower bounds of an HR of the subject over a predetermined duration, where the predetermined duration includes the HR time series window 406. The predetermined duration may be, for example, 10 seconds.

Additionally, each training pair 412 may include additional input data that may increase an accuracy of the HR time series prediction model 420, such as one or more extracted statistical features extracted by feature extractor 404, one or more classifications generated by classifiers 405, and/or one or more parameters 407 of the CT imaging system associated with an HR time series window 406. Further, as described in greater detail below, each training pair 412 may include a confidence level, where the confidence level indicates a desired confidence of the HR time series prediction model 420 that an output of the HR time series prediction model 420 is correct.

A first portion of training pairs 412 may be assigned to a training dataset, a second portion of training pairs 412 may be assigned to a validation dataset, and a third portion of training pairs 412 may be assigned to a test dataset. In an embodiment, training pairs 412 may be assigned to either the training dataset, the validation dataset, or the test dataset randomly in a pre-established proportion. For example, training pairs 412 may be assigned to either the training dataset or the test dataset randomly such that 90% of the training pairs are assigned to the training dataset, and 10% of the training pairs are assigned to the test dataset and the validation dataset. It should be appreciated that the examples provided herein are for illustrative purposes, and the training pairs may be assigned to the training, validation, and test datasets via a different procedure and/or in a different proportion without departing from the scope of this disclosure.

HR time series prediction model 420 may be trained on training pairs 412 to learn to predict upper and lower thresholds of the HR from a preceding HR time series window 406. HR time series prediction model 420 may be stored in an ML model module 442 of the HR prediction system, such as ML module 308 of HR prediction system 302 of FIG. 3. An example method for training HR time series prediction model 420 is described in further detail below with respect to FIG. 5.

HR time series prediction model training system 400 may include a validator 422 that validates the performance of the HR time series prediction model 420 using training pairs of the validation dataset and the test dataset. Validator 422 may use the validation dataset to prevent overfitting, and may output an assessment of a performance of the trained or partially trained HR time series prediction model 420 on the test dataset of training pairs. Training may be completed when HR time series prediction model 420 achieves a minimum error rate in detecting segments. After HR time series prediction model 420 is validated, HR time series prediction model 420 may be fully trained. Trained HR time series prediction model 430 (e.g., the validated HR time series prediction model 420) may be used to predict upper and lower threshold values of an HR of a patient of an imaging system (e.g., imaging system 200), based on HR data collected from the patient over a preceding time window. Trained HR time series prediction model 430 may be deployed in an inference module 444 of the HR prediction system (e.g., inference module 312 of FIG. 3).

It should be appreciated that various medical facilities utilize slightly different scan procedures and thus, an operation of a CT imaging system may be dependent on a particular site in which the CT imaging system is installed. For example, different sites utilize their own patient preparation and breathing protocols. In addition, heart behavior characteristics of patients may vary depending upon factors related to their native geographical location. Thus, HR time series prediction model 430 may be trained on data specific to a given CT imaging system.

FIG. 4B shows a block diagram 450 of a deployment scenario of trained HR time series prediction model 430, where trained HR time series prediction model 430 is used by the HR prediction system to determine a target scanning window 462 used to configure a CT imaging system (e.g., imaging system 200 of FIG. 2) for performing a cardiac scan on a patient 452.

Prior to performing the cardiac scan, HR time series data 456 of patient 452 may be collected by an HR monitoring device 454, such as, for example, an EKG of the imaging system (e.g., EKG 250). From HR time series data 456, time series window generator 403 of FIG. 4A may extract a plurality of overlapping HR time series windows 458, similar to the HR time series windows 406 extracted from stored HR time series data 402 described above. The overlapping HR time series windows 458 may be inputted into trained HR time series prediction model 430 at the predefined inference intervals. As described above in reference to FIG. 4A, additional data generated from and/or associated with the overlapping HR time series windows 458 may be included as inputs into the trained HR time series prediction model 430. For example, CT imaging system parameters 407 used for the cardiac scan, classifications of the patient into one or more categories based on the HR time series data 456, and/or statistical features extracted from the overlapping HR time series windows 458 may be additional inputs into the trained HR time series prediction model 430, and the trained HR time series prediction model 430 may generate an output based on the overlapping HR time series windows 458 and the additional inputs.

At each predefined time interval, trained HR time series prediction model 430 may output a predicted upper and lower threshold HR 460, where the predicted upper and lower threshold HR define boundaries of a predicted HR range of patient 452. Additionally, a confidence level 472 specified by a radiologist 470 may be an additional input into trained HR time series prediction model 430. The confidence level may be a threshold level of confidence with which trained HR time series prediction model 430 produces a predicted upper and lower threshold HR. The confidence level may depend on a target anatomy of the heart of the patient being scanned, and how much the target anatomy is expected to move during an exam. The confidence level is described in greater detail below in reference to FIGS. 5 and 6.

From predicted upper and lower threshold HR 460, a timing of a cardiac cycle and phases of the cardiac cycle may be determined. From the cardiac cycle, a target scanning window 462 (e.g., target scanning duration 752) may be generated, where target scanning window 462 represents a prescribed range of times (e.g., prescribed cardiac phase range 750) within a desired portion of the cardiac cycle (e.g., mid-diastole, end-systole, etc.), when the motion of the heart may be minimized. An imaging system configuration 464 may then be performed, to configure the CT imaging system to image the target anatomies of the heart of patient 452 during target scanning window 462. In some examples, a first timing a start of an X-ray exposure may be scheduled based on the predicted upper threshold HR 460, and a second timing of an end of the X-ray exposure may be scheduled based on the predicted lower threshold HR 460.

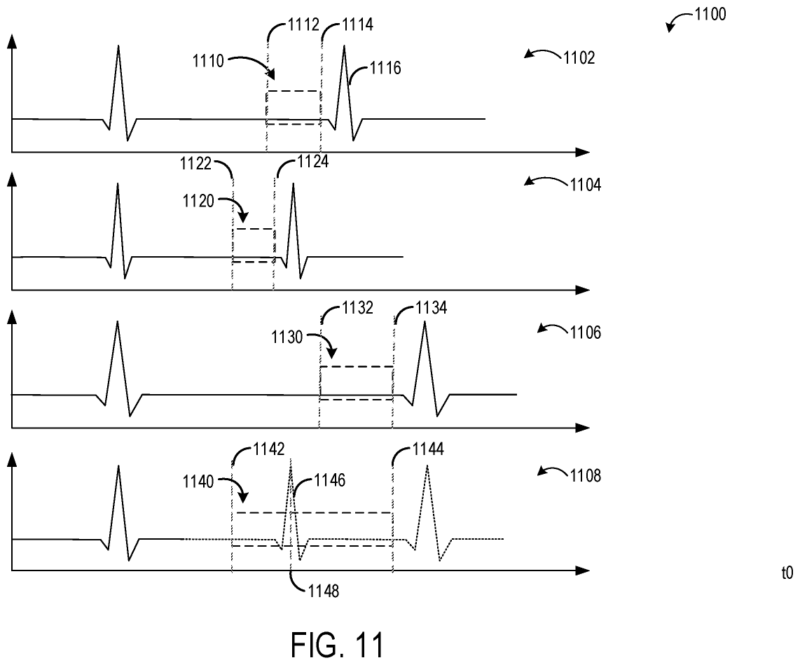

For example, if a predicted lower threshold HR is 55 BPM and a predicted upper threshold HR is 70 BPM, then the exposure may be programmed to start at a first time within a cardiac cycle to account for a heart rate as fast as 70 BPM, and to end at a second time within the cardiac cycle to account for a heart rate as slow as 55 BPM. As a result, a total exposure time may longer than if the heart rate was forecasted to be 60 BPM (e.g., a higher dose), but the exposure may be more robust in the sense that the desired phases may be acquired given heart rates as slow as 55 BPM or as fast as 70 BPM. Additionally, an mA modulation profile of the X-ray exposure may be similarly adapted to cover the range of potential heart beat durations of 55-70 BPM. The scheduling of the exposure based on the upper and lower threshold HRs is described in greater detail below in reference to FIGS. 6 and 11.

Referring now to FIG. 5, a method 500 is shown for training a HR time series prediction model, such as HR time series prediction model 420 of FIG. 4A, according to an exemplary embodiment. The HR time series prediction model may be trained in accordance with the HR time series prediction model training system 400. In some embodiments, the HR time series prediction model may be a deep neural network with a plurality of hidden layers. For example, the HR time series prediction model may be a convolutional neural network (CNN). In other embodiments, the HR time series prediction model may be a recurrent neural network (RNN), where some outputs of nodes of the RNN are used to affect subsequent inputs into the nodes. In still other embodiments, the HR time series prediction model may be a different type of artificial neural network.

Method 500 and the other methods described herein may be executed by a processor of a HR prediction system, such as HR prediction system 302 of FIG. 3. Operations of method 500 may be stored in non-transitory memory of the HR prediction system (e.g., in a training module such as the training module 310 of the HR prediction system 302 of FIG. 3) and executed by a processor of the HR prediction system (e.g., processor 304). The HR time series prediction model may be trained on training data generated as described in reference to FIG. 4A. In some embodiments, the training data may comprise HR time series data.

Method 500 begins at 502, where method 500 includes receiving HR time series data of a plurality of subjects. In some embodiments, the HR time series data may be generated by a source such as an EKG or patient monitor positioned on a patient of an imaging system or healthcare system, and stored in the HR prediction system (e.g., HR time series data 314). Additionally or alternatively, the HR time series data may be collected from a different set of subjects in a different manner.

At 504, method 500 includes generating (e.g., extracting data values of the HR time series data into) a plurality of overlapping time series windows (e.g., HR time series windows 406 of FIG. 4A) of a predefined time. For example, the predefined time may be 10 seconds. The time series windows may be created as described above in reference to FIG. 4A.

For example, a first time series window of 10 seconds may be created from the HR time series data. The first time series window may include 100 individual data values (e.g., a blood pressure, or a voltage) starting with a first data value and ending with a 100th data value of the HR time series data. Each data value may be collected from a corresponding subject at the predetermined time interval. A second time series window of 10 seconds may be created from the HR time series data including 100 data values, starting with a second data value and ending with an 101^{st} data value of the HR time series data, such that the second time series window is offset from the first time series window by one data value, and overlaps the first time series window when plotted on a timeline. A third time series window of 10 seconds may be created from the HR time series data including 100 data values, starting with a third data value and ending with an 103^{th} data value of the HR time series data, such that the third time series window is offset from the second time series window by one data value and from the first time series window by two data values, and overlaps the first time series window and the second time series window when plotted on the timeline, and so on.

An example of the extraction and processing of time series windows during training is shown in FIGS. 9 and 10. Referring to FIG. 9, a time series diagram 900 shows time series HR data of a subject plotted on a line 902. A voltage generated by an HR monitor (e.g., EKG 250 and/or EKG 336) is represented on the Y axis, and time in seconds is represented on the X axis. A depicted portion of the time series HR data includes a first heartbeat 904, and a second heartbeat 906. The time series HR data may be collected continuously from the subject over a period of time. For example, the subject may be a patient, and the time series HR data may be collected from the patient while an imaging study is performed on the patient.

Individual HR data (e.g., voltage) values may be extracted at regular intervals from the time series HR data, to generate an HR data array 908 that may be continuously added to over time. In FIG. 9, a first exemplary data value 910 (e.g., 0.9 mV) corresponds to a time of first heartbeat 904, and a second exemplary data value 912 (e.g., 0.9 mV) corresponds to a time of second heartbeat 906. The extracted data values may then be further extracted into a plurality of overlapping time series windows, as shown in FIG. 10.

FIG. 10 depicts three overlapping time series windows extracted from HR data array 908, including a first time series window 1002, a second time series window 1004, and a third time series window 1006. The time series windows 1002, 1004, and 1006 may be offset from each other by a predefined interval. In FIG. 10, the predefined interval is equal to a second interval at which the HR data values are extracted from the time series data (e.g., shown by line 902 of FIG. 9). In other embodiments, a predefined interval of a different length may be used. For example, the HR time series data of FIG. 9 may be collected every 0.1 seconds, and time series windows 1002, 1004, and 1006 may be offset from each other by 0.2 seconds, or 0.3 seconds, or a different amount of time.

At the passage of each predefined interval, a new time series window may be inputted into a HR time series prediction model, as described above in reference to FIG. 4A. The HR time series prediction model may learn to perform sliding window inferences on the HR data included in the time series windows over various inference intervals, where a single time series window of HR data is inputted into the HR time series prediction model at each inference interval.

For example, at a first inference interval, a first data value (e.g., voltage) of HR data array 908 may be assigned to a first position of a first time series window 1002. At a second inference interval, a second data value of HR data array 908 may be assigned to a second position of first time series window 1002. Additionally, the second data value may be assigned to a first position of a second time series window 1004. At a third inference interval, a third data value of HR data array 908 may be assigned to a third position of first time series window 1002, a second position of second time series window 1004, and a first position of a third time series window 1006, and so on. In this way, a plurality of overlapping time series windows of HR time series data may be extracted from HR data array 908, where each overlapping time series window is offset from a previous time series window and a subsequent time series window by the inference interval.

When the predefined number of data values of an overlapping time series window has been obtained, meaning that a data value is included in each position of the relevant time series window, the relevant time series window may be inputted into the HR time series prediction model. A first dashed line 1010 shows a completion of first time series window 1002. A second dashed line 1012 shows a completion of second time series window 1004, and a third dashed line 1014 shows a completion of third time series window 1006. When each time series window includes the predefined number of data values, the time series window may be inputted into the HR time series prediction model. After the passage of a subsequent inference interval, a next time series window may be inputted into the HR time series prediction model, and so on.

Returning to method 500, at 506, method 500 includes extracting one or more statistical features of the overlapping time series windows. The statistical features may include ECG waveform features, such as an amplitude and/or time of an R-wave (R-peak) of a time series window; an amplitude and/or time of a p-wave of the time series window; an amplitude and/or time of a t-wave of the time series window; a slope of a QRS complex of the time series window; a quantification of electrocardiograph (EKG) noise in the time series window using a Fourier analysis or other general signal processing techniques; and/or other statistical features (e.g., mean, median, or standard deviation data, etc.). The ECG waveform features may be generated by an 3rd party ECG monitor or R-peak trigger device, or they may be generated by custom feature extraction modes to detect the desired ECG waveform features.

At 508, extracting the one or more statistical features may include performing a classification of a time series window using a classification model of the CT imaging system. The classification model may be a third-party classification model used by the CT imaging system to identify different types of patients. For example, the classification model may detect an abnormal heart rhythm in the time series window, such as an A-fib, bigeminy/trigeminy, etc., and may classify the time series window based on the abnormal heart rhythm.

At 510, method 500 includes generating a plurality of training pairs, where each training pair of the plurality of training pairs includes at least an array of data values corresponding to a time series window, as input data, and a corresponding target upper and lower threshold HR of the subject as ground truth data. The input data may also include one or more statistical features extracted from a relevant time series window, including one or more classifications of the relevant time series window as described above. The input data may also include one or more parameters associated with the relevant time series window, such as, for example, whether a contrast agent was administered to a patient of the time series window, a type of breath holding technique used by the patient, or a different parameter. The statistical features, classifications, and parameters may be inputted into the time series prediction model as binary or numerical encodings. For example, a first encoding may be used for a first breath holding technique; a second encoding may be used for a second breath holding technique; a third encoding may be used for a third breath holding technique; and so on. Additionally, each training pair may include a confidence level specified by an operator of the CT imaging system (e.g., radiologist 470). The HR time series prediction model may learn to reduce or increase a difference between the upper and lower threshold HR values based on the confidence level. In other embodiments, additional or different patient data may be included in the input data, such as, for example, history data, test results, ejection fraction, cardiac health information, and/or age/sex/demographic data of the patient.

For example, a first training pair may include a first HR time series window, the additional input data described above, and a confidence level of “low”. A first set of target upper and lower threshold HR values of the subject used as ground truth data for the first HR time series window may establish a first, wider range of predicted HRs of the subject within which the imaging system may be configured to perform a cardiac scan. A second training pair may include the first HR time series window, the additional input data described above, and a confidence level of “high”. A second set of target upper and lower threshold HR values of the subject used as ground truth data for the first HR time series window in the second training pair may establish a second, narrower range of predicted HRs of the subject within which the imaging system may be configured to perform a cardiac scan. Thus, the HR time series prediction model may learn to adjust a range of upper and lower HR threshold values outputted by the HR time series prediction model based on whether the confidence level is lower or higher. If the confidence level is lower, a wider HR range may be outputted by the HR time series prediction model. Alternatively, if the confidence level is higher, a narrower HR range may be outputted by the HR time series prediction model.

In some embodiments, the upper and lower threshold HRs used as ground truth data for different confidence levels may be specified manually, by one or more human experts. For example, a labeling process may be performed where for a given time series window, the human experts may specify, for each confidence level of a plurality of confidence levels, upper and lower threshold HRs that might correspond to the confidence level. For example, for a first time series window, the human expert(s) may specify that at a confidence level of 60%, a plausible upper threshold HR for the time series window may be 90 BPM, and a plausible lower threshold HR for the time series window may be 60 BPM; at a confidence level of 70%, a plausible upper threshold HR for the time series window may be 85 BPM, and a plausible lower threshold HR for the time series window may be 62 BPM; at a confidence level of 80%, a plausible upper threshold HR for the time series window may be 82 BPM, and a plausible lower threshold HR for the time series window may be 65 BPM; and so on, until a desired number of confidence level/upper and lower threshold HRs pairs have been obtained.

Alternatively, in some embodiments, a conventional time-series prediction model may be used to aid in the generation of the confidence level/upper and lower threshold HRs pairs. The conventional time-series prediction model could be trained on real HR data of a plurality of patients (e.g., the same stored HR time series data 402 used to generate the time series windows), using real subsequent HR data as ground truth data. The trained conventional time series prediction model may then be used to predict a next cardiac cycle for each time series window of the plurality of time series windows, and derive a predicted HR based on the next cardiac cycle. Based on the predicted HR, plausible upper and lower threshold HRs may be randomly assigned to the time series windows, for example, using a rules-based system, that cover a variety of HR ranges, including narrow and wide HR ranges. For each time series window, the randomly assigned upper and lower threshold HRs may then be evaluated by the human experts, who could assign a probability (e.g., a confidence level) that an X-ray exposure performed within the randomly assigned HR range would capture a desired portion of a heart during a desired/requested phase of a cardiac cycle.

Once the training pairs have been created, the training pairs may be divided into a training dataset, a validation dataset, and a test dataset, as described above in reference to FIG. 4A.

At 512, method 500 includes training the HR time series prediction model on the training pairs. During training, the HR time series prediction model may be configured to iteratively adjust one or more of a plurality of weights of the HR time series prediction model in order to minimize a loss function, based on a difference between an output of the HR time series prediction model for a given time series window, confidence level, and the corresponding target upper and lower HR assigned to the time series window as ground truth target data for the relevant training pair. In one embodiment, the loss function is a Mean Absolute Error (MAE) loss function. It should be appreciated that the examples provided herein are for illustrative purposes, and other types of loss functions may be used without departing from the scope of this disclosure.

The difference (or loss), as determined by the loss function, may be back-propagated through the HR time series prediction model to update the weights (and biases) of the hidden layers. In some embodiments, back propagation of the loss may occur in accordance with a gradient descent algorithm, wherein a gradient of the loss function (a first derivative, or approximation of the first derivative) is determined for each weight and bias of the HR time series prediction model. Each weight (and bias) of the HR time series prediction model is then updated by adding the negative of the product of the gradient determined (or approximated) for the weight (or bias) with a predetermined step size. Updating of the weights and biases may be repeated until the weights and biases of the HR time series prediction model converge, or the rate of change of the weights and/or biases of the HR time series prediction model for each iteration of weight adjustment are under a threshold.

Turning now to FIG. 6, an exemplary method 600 is shown for deploying a trained HR time series prediction model, such as trained HR time series prediction model 430 of FIG. 4B, to perform inferences on HR time series data collected from a patient of an imaging system (e.g., imaging system 200 of FIG. 2) to predict upper and lower threshold values of an HR of the patient. The predicted upper and lower threshold values may be used to configure the imaging system to image a heart of the patient during a cardiac scan, during a portion of the subsequent cardiac cycle when the heart of the patient is not moving (e.g., where an amount of motion of the heart is below a threshold amount). In an embodiment, operations of method 600 may be stored in non-transitory memory of the HR prediction system (e.g., in an inference module such as inference module 312) and executed by a processor of the HR prediction system (e.g., processor 304).

Method 600 begins at 602, where method 600 includes collecting HR time series data of the patient for a duration prior to performing a scan of the heart. In various embodiments, the HR time series data may be collected by a patient monitor or EKG positioned on a patient of a healthcare system, as described above. The HR time series data may not be generated by the same patient monitor or EKG used to generate the training data. In other words, a first patient monitor or EKG may be used to generate a first set of HR time series data used to train the HR time series prediction model, and a second patient monitor or EKG may be used to generate a second set of HR time series data for the performance of method 600. In other embodiments, the HR time series data may be generated by a different device other than a patient monitor or an EKG.

At 604, method 600 includes extracting statistical features of the HR time series data. The extracted statistical features may include, for example, waveform features such as R-peak, p-wave, t-wave, slope, electrocardiogram (ECG) noise, etc. The statistical features may be inputs into the HR time series prediction model, as described above.

In some embodiments, extracting statistical features of the HR time series data may include performing a classification of the HR time series data. For example, the classification of the HR time series data may be performed using one or more classification models of the CT imaging system. In some embodiments, the classification may be based on a detected abnormal heart beat (e.g., an A-fib, bigeminy/trigeminy, etc.). The classification may be additional inputs into the HR time series prediction model.

At 606, method 600 includes receiving a confidence level for the upper and lower threshold values of the HR outputted by the trained HR time series prediction model. In various embodiments, the confidence level is specified by an operator of the imaging system at a time of performing the cardiac scan. The confidence level may reflect a desired probabilistic certainty of the operator of the predicted the upper and lower threshold values of the HR being accurate, for a given clinical task. The confidence level may be a percentage reflecting a threshold probabilistic certainty of the predicted the upper and lower threshold values of the HR being accurate, such as 60%, 70%, 90%, etc. In some embodiments, the confidence level may be specified as a category, based on gradations between a lowest and a highest confidence level (e.g., low, medium, high, etc.), or the confidence level may be specified in a different manner. The confidence level may indicate to the trained HR time series prediction model whether a narrow HR range is desired, where an importance of the motion of the heart being minimized is higher, or a wider HR range is desired, where an importance of the motion of the heart being minimized is lower.

At 608, method 600 includes predicting an upper threshold HR value and a lower threshold HR value of the patient, based on the HR time series data collected from the patient, the extracted statistical features, classifications of the HR time series data, parameters of the CT imaging system associated with the HR time series data, and the received confidence level, using the trained HR time series prediction model. As described above, HR time series windows of a predefined length (e.g., time) may be extracted from the HR time series data, and the HR time series windows may be inputted into the trained HR time series prediction model along with the corresponding confidence level and other input data.

In some embodiments, additional patient data may also be used to predict the upper and lower threshold HR values. For example, patient history, test results, demographic data of the patient, or other patient data may be additional inputs into the trained HR time series prediction model, and the trained HR time series prediction model may predict the upper threshold HR value and the lower threshold HR value of the patient based on the additional patient data.

At 610, method 600 includes performing a cardiac scan on the patient, adjusting a dose and/or timing (e.g., scheduling) based on the predicted upper and lower threshold HR values. The predicted upper and lower threshold HR values may be updated immediately prior to performing the cardiac scan. For example, in some embodiments, a prescribed dose and/or timing may be retrieved from a lookup table based on the predicted upper and lower threshold HR values. In other embodiments, the prescribed dose may be predicted based on the predicted upper and lower threshold HR values, for example, using statistical methods and/or other predictive models.

As an example, FIG. 11 shows an X-ray exposure comparison 1100, including four plots of a cardiac cycle during a scan of a patient using a CT imaging system such as the CT imaging system 100 of FIG. 1. A first plot 1102 illustrates a first exemplary exposure 1110, which starts at a first time 1112 and ends at a second time 1114. First exemplary exposure 1110 corresponds to an X-ray exposure for a nominal (e.g., expected) heart rate, where first exemplary exposure 1110 may be expected to image a portion of a heart of a patient with a 70-80% probability of capturing the portion at a desired or requested phase of the cardiac cycle (e.g., mid-diastole/end systole, prior to a heartbeat 1116).

A second plot 1104 illustrates a second exemplary exposure 1120, which starts at a first time 1122 and ends at a second time 1124. Second exemplary exposure 1120 corresponds to an X-ray exposure for a faster heart rate (e.g., with respect to the nominal heart rate of plot 1102), where second exemplary exposure 1120 may be expected to image the portion of the heart with the 70-80% probability of capturing the portion at the desired or requested phase of a shorter cardiac cycle. In other words, if the faster heart rate is expected, second exemplary exposure 1120 may be selected rather than first exemplary exposure 1110. Because the cardiac cycle is shorter, less time may be available for second exemplary exposure 1120 than for first exemplary exposure 1110, and second exemplary exposure 1120 may start at an earlier time than first exemplary exposure 1110.

A third plot 1106 illustrates a third exemplary exposure 1130, which starts at a first time 1132 and ends at a second time 1134. Third exemplary exposure 1130 corresponds to an X-ray exposure for a slower heart rate than the nominal heart rate of plot 1102, where third exemplary exposure 1130 may be expected to image the portion of the heart with the 70-80% probability of capturing the portion at the desired or requested phase of a longer cardiac cycle. In other words, if the slower heart rate is expected, third exemplary exposure 1130 may be selected rather than first exemplary exposure 1110 or second exemplary exposure 1120. Because the cardiac cycle is longer, more time may be available for third exemplary exposure 1130 than for first exemplary exposure 1110, and third exemplary exposure 1130 may start at a later time than first exemplary exposure 1110.

A fourth plot 1108 illustrates a fourth exemplary exposure 1140, which is timed to image the portion of the heart with the 70-80% probability of capturing the portion at the desired or requested phase of the cardiac cycle in both the case of a shorter-than-expected heart rate and in the case of a longer-than-expected heart rate. Thus, fourth exemplary exposure 1140 starts at a first time 1142 and ends at a second time 1144, where first time 1142 may be equal to first time 1122 of second exemplary exposure 1120, and second time 1144 may be equal to second time 1134 of third exemplary exposure 1130. In other words, fourth exemplary exposure 1140 may be scheduled to start based on a fastest predicted heart rate (e.g., a predicted upper threshold HR), and fourth exemplary exposure 1140 may be scheduled to end based on a slowest predicted heart rate (e.g., a predicted lower threshold HR). If a subsequent heartbeat 1146 occurs prior to the second time 1144 when the fourth exemplary exposure 1140 is scheduled to end, the fourth exemplary exposure 1140 may be advantageously ended early at a time 1148, to minimize the corresponding dose administered to the patient. In this way, capturing the portion at the desired or requested phase of the cardiac cycle within a desired probability may be ensured for a wider range of heart rate variance than may be achieved using first exemplary exposure 1110.

Further, the desired probability of capturing the portion at the desired or requested phase of the cardiac cycle may be adjusted by an operator of the CT imaging system. In some cases, the operator may specify that the fourth exemplary exposure 1140 be timed to image the portion of the heart with a lower probability, such as a 60% probability of capturing the portion at the desired or requested phase of the cardiac cycle. The operator may specify the lower probability as a confidence level to an HR prediction system such as HR prediction system 302, and predicted upper and lower threshold HRs corresponding to the lower probability may be outputted by the HR prediction system. The predicted upper and lower threshold HRs may then be used to adjust the first time 1142 and the second time 1144 accordingly. By specifying the lower probability, a range of HRs between the predicted upper and lower threshold HRs may be increased, thereby increasing a duration of fourth exemplary exposure 1140.

Alternatively, the operator may specify that the fourth exemplary exposure 1140 be timed to image the portion of the heart with a higher probability, such as a 90% probability of capturing the portion at the desired or requested phase of the cardiac cycle. The operator may specify the higher probability as a higher confidence level to the HR prediction system and predicted upper and lower threshold HRs corresponding to the higher probability may be outputted by the HR prediction system. The predicted upper and lower threshold HRs may then be used to adjust the first time 1142 and the second time 1144 accordingly. By specifying the higher probability, the range of HRs between the predicted upper and lower threshold HRs may be reduced, thereby decreasing the duration of fourth exemplary exposure 1140.

Thus, the dose may be calculated based on a best, worst or nominal case. For instance, the best case (e.g., lowest dose) is if the actual heart rate is the same as the upper threshold HR value. In this case, the exposure may start early enough for the highest forecasted heart rate. When an R-peak is detected, the exposure mA profile and exposure end time may be updated based on the actual (high) heart rate. Alternatively, the dose may be predicted based on a worst-case scenario (e.g., highest dose), where the exposure starts early enough for the upper threshold HR value, and ends based on the lower threshold HR value, thus creating the longest and highest dose exposure.

As yet another alternative, the prediction could be based on a nominal (expected) heart rate forecast, where the exposure may be scheduled to start based on the higher threshold HR value, and end based on the nominally forecasted HR shown in plot 1102 (e.g., starting at first time 1122, and ending at second time 1114). An advantage of basing the prediction on the nominal heart rate forecast in this manner is that the dose may be reduced, with respect to the worst case scenario described above.

In some examples, a start time of a planned X-ray exposure may be set based on the upper threshold HR value, and an end time of the planned exposure may be set based on the lower threshold HR value. The end of the exposure can be updated in real time based on an actual heart rate, In other words, if a heartbeat is faster than predicted, then the X-ray exposure can updated based on the actual HR detected (e.g., the exposure ends early).

It should be appreciated that the predicted upper and lower threshold HR values may be predicted using the HR prediction model at various times. For example, the values may be predicted during a patient scan prescription for a dose prediction, or during the scan in real time, or both. The values may be predicted a plurality of times during preparing for a scan and performing the scan.

At 612, method 600 includes reconstructing an image from projection data acquired from the cardiac scan, and displaying the reconstructed image on a display device of the imaging system (e.g., display device 232 of imaging system 200) and/or saving the reconstructed image in a memory of the imaging system. Method 600 ends.

Thus, systems and methods are proposed to more accurately predict a desired phase(s) of a heart cycle to minimize x-ray exposure to patients during prospectively-gated cardiac CT studies. The desired phase of the heart cycle can be more accurately predicted based on HR time series data collected over a duration during a pre-scan period, using an ML model, than by other predictive techniques based on heartbeat timing. Current methods typically rely on predicting an average HR of a patient and adding an amount of padding (i.e. HR variation allowance) to adjust for uncertainty. However, the current methods incorrectly assume an equal probability of having a faster or slower heartbeat, which reduces an accuracy of the current methods and leads to an increased number of failed and/or repeated scans, exposing patients to increased radiation. In contrast, as provided herein, an HR time series prediction model may predict an upper HR threshold value and a lower HR threshold value, which may be used to set scan parameters (e.g. table speed, gantry speed and slice thickness) and start exposure and end exposure times for the scan. By basing the scan parameters and start/end exposure times on the upper and lower HR threshold values, the assumption of the equal probability of having a faster or slower heartbeat is avoided, resulting in scan parameters and start and end exposure times with a higher probability of being in a range where a motion of the heart is minimized. As a result, the number of failed and/or repeated scans may be reduced, increasing a health and safety of the patients. Further, an availability of imaging resources is increased for use with other patients, leading to increased efficiency of a use of the CT imaging system.

Additionally, the HR time series prediction model may take as an additional input a desired statistical confidence level that the predicted upper and lower HR thresholds will be correct, where the desired statistical confidence level may be provided by an operator of the CT imaging system. The ML model may output the predicted upper and lower HR thresholds based on the desired confidence level. Thus, rather than setting a padding around a predicted HR to account for uncertainty, a user of a CT imaging system can set the desired confidence level for a clinical task, and the CT imaging system calculates a corresponding dose impact corresponding to the upper and lower HR thresholds based on the desired confidence level. In this way, a narrower time window for performing a cardiac scan may be selected when an importance of the heart being motionless is higher (e.g., for smaller and more active anatomies), and a wider time window for performing the cardiac scan may be selected when the importance of the heart being motionless is lower (e.g., for larger, more stable anatomies), resulting in a lower probability of failed scans.

The technical effect of basing scan parameters and start/end exposure times for a cardiac scan using a CT imaging system on upper and lower HR threshold values predicted by an ML model, where the upper and lower HR threshold values are predicted based on a desired confidence level of an output of the ML model, is that a number of failed cardiac scans may be reduced.

The disclosure also provides support for a method for a computed tomography (CT) imaging system, the method comprising: predicting an upper threshold value and a lower threshold value of a heart rate (HR) of a patient of the CT imaging system, based on HR time series data collected from the patient over a duration and a confidence level specified by an operator of CT imaging system, configuring the CT imaging system to perform a cardiac scan during a cardiac phase range, based on the upper threshold value and the lower threshold value, performing the cardiac scan, reconstructing an image based on data acquired during the cardiac scan, and displaying the image on a display device of the CT imaging system and/or storing the image in a memory of the CT imaging system. In a first example of the method, the method further comprises: predicting the upper threshold value and the lower threshold value of the HR of the patient based on the HR time series data and the confidence level using an HR prediction model, where the HR prediction model comprises a machine learning (ML) model trained on stored HR time series data acquired from a plurality of human subjects. In a second example of the method, optionally including the first example, the plurality of human subjects includes subjects having a healthy heart and subjects suffering from a heart condition. In a third example of the method, optionally including one or both of the first and second examples, the HR prediction model comprises one of a convolutional neural network (CNN) and a recurrent neural network (RNN). In a fourth example of the method, optionally including one or more or each of the first through third examples, the stored HR time series data used to train the HR prediction model includes at least one of: one or more statistical features extracted from samples of the stored HR time series data, one or more parameters of the CT imaging system associated with the stored HR time series data, patient history, test results, and/or demographic data of a patient of the stored HR time series data. In a fifth example of the method, optionally including one or more or each of the first through fourth examples, the one or more statistical features extracted from the samples of the stored HR time series data includes at least one of an amplitude and/or time of an R-wave (R-peak), an amplitude and/or time of a p-wave, an amplitude and/or time of a t-wave, a slope of a QRS complex, a quantification of electrocardiograph (EKG) noise, and a classification of a sample performed by a classification model of the CT imaging system. In a sixth example of the method, optionally including one or more or each of the first through fifth examples, the classification is based on the classification model detecting an abnormal heart rhythm in the stored HR time series data. In a seventh example of the method, optionally including one or more or each of the first through sixth examples, the one or more parameters of the CT imaging system associated with the stored HR time series data includes at least one of an indication of whether a contrast agent was administered during a collection of samples of the stored HR time series data, one or more contrast injection parameters, and/or a breath hold duration/parameters of a patient during the collection of samples. In a eighth example of the method, optionally including one or more or each of the first through seventh examples, predicting the upper threshold value and the lower threshold value of the HR of the patient based on the HR time series data collected from the patient and the confidence level further comprises: extracting the one or more statistical features from the HR time series data collected from the patient, and predicting the upper threshold value and the lower threshold value of the HR of the patient based on the HR time series data collected from the patient over the duration, the specified confidence level, and one or more of an extracted statistical feature and a parameter of the CT imaging system associated with the HR time series data. In a ninth example of the method, optionally including one or more or each of the first through eighth examples, the confidence level is specified as a percentage indicating a threshold probabilistic certainty of the predicted the upper and lower threshold values of the HR being sufficiently accurate to configure the cardiac scan to be performed during the cardiac phase range. In a tenth example of the method, optionally including one or more or each of the first through ninth examples, the confidence level is specified by selecting a category of a plurality of categories corresponding to gradations between a lowest threshold probabilistic certainty of the predicted the upper and lower threshold values of the HR being sufficiently accurate to configure the cardiac scan to be performed during the cardiac phase range. and a highest threshold probabilistic certainty of the predicted the upper and lower threshold values of the HR being sufficiently accurate to configure the cardiac scan to be performed during the cardiac phase range. In an eleventh example of the method, optionally including one or more or each of the first through tenth examples, configuring the CT imaging system to perform the cardiac scan based on the upper threshold value and the lower threshold value further comprises scheduling a start of an X-ray exposure based on the upper threshold value, and scheduling an end of the X-ray exposure based on the lower threshold value. In a twelfth example of the method, optionally including one or more or each of the first through eleventh examples, the cardiac phase range is prescribed by the operator, and the cardiac phase range is one of a middle of diastole of a cardiac cycle and an end of systole of the cardiac cycle. In a thirteenth example of the method, optionally including one or more or each of the first through twelfth examples, the method further comprises: updating a prediction of the upper threshold value and the lower threshold value of the HR of the patient immediately prior to performing the cardiac scan.

The disclosure also provides support for a computed tomography (CT) imaging system, comprising: an electrocardiograph (EKG), a processor, and a memory storing instructions that when executed, cause the processor to: collect heart rate (HR) time series data from a patient of the CT imaging system over a duration, via the EKG, extract one or more statistical features of the collected HR time series data, receive a confidence level specified by an operator of the CT imaging system, the confidence level a desired probabilistic certainty that a prediction of an HR of the patient generated by the CT imaging system is accurate, receive a cardiac phase range specified by the operator for performing a cardiac scan of the patient using the CT imaging system, predict an upper threshold value and a lower threshold value of the HR of the patient, using a machine learning (ML) model, based on the HR time series data, the extracted one or more statistical features, and the specified confidence level, adjust a dose of radiation and a timing of a CT scan performed using the CT imaging system based on the received cardiac phase range and the predicted upper threshold value and lower threshold value, perform the cardiac scan on the patient at the adjusted timing using the adjusted dose, reconstruct an image based on data acquired during the cardiac scan, and display the image on a display device of the CT imaging system and/or store the image in a memory of the CT imaging system. In a first example of the system, the system further comprises: an HR prediction system, wherein the upper threshold value and the lower threshold value of the HR of the patient are predicted by an HR time series prediction model of the HR prediction system. In a second example of the system, optionally including the first example, the extracted one or more statistical features include at least one of an amplitude and/or time of an R-wave (R-peak), an amplitude and/or time of a p-wave, an amplitude and/or time of a t-wave, a slope of a QRS complex, a quantification of electrocardiograph (EKG) noise, and a classification of the HR time series data performed by a classification model of the CT imaging system. In a third example of the system, optionally including one or both of the first and second examples, the HR time series prediction model is one of a convolutional neural network (CNN) and a recurrent neural network (RNN).

The disclosure also provides support for a method for a computed tomography (CT) imaging system, the method comprising: collecting heart rate (HR) time series data from a patient of the CT imaging system over a duration, extracting statistical features from the HR time series data, performing a classification of the HR time series data using a classification model of the CT imaging system, receiving a confidence level specified by an operator of the CT imaging system, the confidence level a desired probabilistic certainty of an accuracy of a prediction of an HR of the patient generated by the CT imaging system, predicting an upper threshold value and a lower threshold value of the HR of the patient using a machine learning (ML) model, the ML model taking as input the HR time series data, the specified confidence level, the extracted statistical features, and the classification, adjusting a dose of radiation and a timing of a cardiac scan of the patient based on the predicted upper threshold value and lower threshold value, performing the cardiac scan on the patient at the adjusted timing using the adjusted dose, and displaying an image reconstructed from data acquired during the cardiac scan and/or storing the image in a memory of the CT imaging system. In a first example of the method, adjusting the dose of radiation and the timing of the cardiac scan of the patient based on the predicted upper threshold value and lower threshold value further comprises timing a start of an X-ray exposure based on the upper threshold value, and timing an end of the X-ray exposure based on the lower threshold value.

When introducing elements of various embodiments of the present disclosure, the articles “a,” “an,” and “the” are intended to mean that there are one or more of the elements. The terms “first,” “second,” and the like, do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (e.g., a material, element, structure, member, etc.) can be connected to or coupled to another object regardless of whether the one object is directly connected or coupled to the other object or whether there are one or more intervening objects between the one object and the other object. In addition, it should be understood that references to “one embodiment” or “an embodiment” of the present disclosure are not intended to be interpreted as excluding the existence of additional embodiments that also incorporate the recited features.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative and should not be construed to be limiting in any manner.

1. A method for a computed tomography (CT) imaging system, the method comprising:
predicting an upper threshold value and a lower threshold value of a heart rate (HR) of a patient of the CT imaging system, based on HR time series data collected from the patient over a duration and a confidence level specified by an operator of CT imaging system;
configuring the CT imaging system to perform a cardiac scan during a cardiac phase range, based on the upper threshold value and the lower threshold value;
performing the cardiac scan;
reconstructing an image based on data acquired during the cardiac scan; and
displaying the image on a display device of the CT imaging system and/or storing the image in a memory of the CT imaging system.

2. The method of claim 1, further comprising predicting the upper threshold value and the lower threshold value of the HR of the patient based on the HR time series data and the confidence level using an HR prediction model, where the HR prediction model comprises a machine learning (ML) model trained on stored HR time series data acquired from a plurality of human subjects.

3. The method of claim 2, wherein the plurality of human subjects includes subjects having a healthy heart and subjects suffering from a heart condition.

4. The method of claim 2, wherein the HR prediction model comprises one of a convolutional neural network (CNN) and a recurrent neural network (RNN).

5. The method of claim 2, wherein the stored HR time series data used to train the HR prediction model includes at least one of:
one or more statistical features extracted from samples of the stored HR time series data;
one or more parameters of the CT imaging system associated with the stored HR time series data;
patient history, test results, and/or demographic data of a patient of the stored HR time series data.

6. The method of claim 5, wherein the one or more statistical features extracted from the samples of the stored HR time series data includes at least one of an amplitude and/or time of an R-wave (R-peak), an amplitude and/or time of a p-wave, an amplitude and/or time of a t-wave, a slope of a QRS complex, a quantification of electrocardiograph (EKG) noise, and a classification of a sample performed by a classification model of the CT imaging system.

7. The method of claim 6, wherein the classification is based on the classification model detecting an abnormal heart rhythm in the stored HR time series data.

8. The method of claim 5, wherein the one or more parameters of the CT imaging system associated with the stored HR time series data includes at least one of an indication of whether a contrast agent was administered during a collection of samples of the stored HR time series data, one or more contrast injection parameters, and/or a breath hold duration/parameters of a patient during the collection of samples.

9. The method of claim 5, wherein predicting the upper threshold value and the lower threshold value of the HR of the patient based on the HR time series data collected from the patient and the confidence level further comprises:
extracting the one or more statistical features from the HR time series data collected from the patient, and predicting the upper threshold value and the lower threshold value of the HR of the patient based on the HR time series data collected from the patient over the duration, the specified confidence level, and one or more of an extracted statistical feature and a parameter of the CT imaging system associated with the HR time series data.

10. The method of claim 1, wherein the confidence level is specified as a percentage indicating a threshold probabilistic certainty of the predicted the upper and lower threshold values of the HR being sufficiently accurate to configure the cardiac scan to be performed during the cardiac phase range.

11. The method of claim 1, wherein the confidence level is specified by selecting a category of a plurality of categories corresponding to gradations between a lowest threshold probabilistic certainty of the predicted the upper and lower threshold values of the HR being sufficiently accurate to configure the cardiac scan to be performed during the cardiac phase range. and a highest threshold probabilistic certainty of the predicted the upper and lower threshold values of the HR being sufficiently accurate to configure the cardiac scan to be performed during the cardiac phase range.

12. The method of claim 1, wherein configuring the CT imaging system to perform the cardiac scan based on the upper threshold value and the lower threshold value further comprises scheduling a start of an X-ray exposure based on the upper threshold value, and scheduling an end of the X-ray exposure based on the lower threshold value.

13. The method of claim 1, wherein the cardiac phase range is prescribed by the operator, and the cardiac phase range is one of a middle of diastole of a cardiac cycle and an end of systole of the cardiac cycle.

14. The method of claim 1, further comprising updating a prediction of the upper threshold value and the lower threshold value of the HR of the patient immediately prior to performing the cardiac scan.

15. A computed tomography (CT) imaging system, comprising:
an electrocardiograph (EKG);
a processor; and
a memory storing instructions that when executed, cause the processor to:
collect heart rate (HR) time series data from a patient of the CT imaging system over a duration, via the EKG;
extract one or more statistical features of the collected HR time series data;
receive a confidence level specified by an operator of the CT imaging system, the confidence level a desired probabilistic certainty that a prediction of an HR of the patient generated by the CT imaging system is accurate;
receive a cardiac phase range specified by the operator for performing a cardiac scan of the patient using the CT imaging system;
predict an upper threshold value and a lower threshold value of the HR of the patient, using a machine learning (ML) model, based on the HR time series data, the extracted one or more statistical features, and the specified confidence level;
adjust a dose of radiation and a timing of a CT scan performed using the CT imaging system based on the received cardiac phase range and the predicted upper threshold value and lower threshold value;
perform the cardiac scan on the patient at the adjusted timing using the adjusted dose;
reconstruct an image based on data acquired during the cardiac scan; and
display the image on a display device of the CT imaging system and/or store the image in a memory of the CT imaging system.

16. The CT imaging system of claim 15, further comprising an HR prediction system, wherein the upper threshold value and the lower threshold value of the HR of the patient are predicted by an HR time series prediction model of the HR prediction system.

17. The CT imaging system of claim 16, wherein the extracted one or more statistical features include at least one of an amplitude and/or time of an R-wave (R-peak), an amplitude and/or time of a p-wave, an amplitude and/or time of a t-wave, a slope of a QRS complex, a quantification of electrocardiograph (EKG) noise, and a classification of the HR time series data performed by a classification model of the CT imaging system.

18. The CT imaging system of claim 16, wherein the HR time series prediction model is one of a convolutional neural network (CNN) and a recurrent neural network (RNN).

19. A method for a computed tomography (CT) imaging system, the method comprising:
collecting heart rate (HR) time series data from a patient of the CT imaging system over a duration;
extracting statistical features from the HR time series data;
performing a classification of the HR time series data using a classification model of the CT imaging system;
receiving a confidence level specified by an operator of the CT imaging system, the confidence level a desired probabilistic certainty of an accuracy of a prediction of an HR of the patient generated by the CT imaging system;
predicting an upper threshold value and a lower threshold value of the HR of the patient using a machine learning (ML) model, the ML model taking as input the HR time series data, the specified confidence level, the extracted statistical features, and the classification;
adjusting a dose of radiation and a timing of a cardiac scan of the patient based on the predicted upper threshold value and lower threshold value;
performing the cardiac scan on the patient at the adjusted timing using the adjusted dose; and
displaying an image reconstructed from data acquired during the cardiac scan and/or storing the image in a memory of the CT imaging system.

20. The method of claim 19, wherein adjusting the dose of radiation and the timing of the cardiac scan of the patient based on the predicted upper threshold value and lower threshold value further comprises timing a start of an X-ray exposure based on the upper threshold value, and timing an end of the X-ray exposure based on the lower threshold value.
